SEQUENCE LISTING (15 sequences)

SEQ ID NO: 1 - protein, 128 aa, synthetic construct
  REGION 1..128: note = Synthetic: CD59 polypeptide
MGIQGGSVLFGLLLVLAVFCHSGHSLQCYNCPNPTADCKTAVNCSSDFDACLITKAGLQVYNKCWKFEHCNFNDVTTRLRENELTYYCCKKDLCNFNEQLENGGTSLSEKTVLLLVTPFLAAAWSLHP

SEQ ID NO: 2 - protein, 77 aa, synthetic construct
  REGION 1..77: note = Synthetic: CD59 polypeptide
LQCYNCPNPTADCKTAVNCSSDFDACLITKAGLQVYNKCWKFEHCNFNDVTTRLRENELTYYCCKKDLCNFNEQLEN

SEQ ID NO: 3 - protein, 500 aa, synthetic construct
  REGION 1..500: note = Synthetic: SERPING1 polypeptide
MASRLTLLTLLLLLLAGDRASSNPNATSSSSQDPESLQDRGEGKVATTVISKMLFVEPILEVSSLPTTNSTTNSATKITANTTDEPTTQPTTEPTTQPTIQPTQPTTQLPTDSPTQPTTGSFCPGPVTLCSDLESHSTEAVLGDALVDFSLKLYHAFSAMKKVETNMAFSPFSIASLLTQVLLGAGENTKTNLESILSYPKDFTCVHQALKGFTTKGVTSVSQIFHSPDLAIRDTFVNASRTLYSSSPRVLSNNSDANLELINTWVAKNTNNKISRLLDSLPSDTRLVLLNAIYLSAKWKTTFDPKKTRMEPFHFKNSVIKVPMMNSKKYPVAHFIDQTLKAKVGQLQLSHNLSLVILVPQNLKHRLEDMEQALSPSVFKAIMEKLEMSKFQPTLLTLPRIKVTTSQDMLSIMEKLEFFDFSYDLNLCGLTEDPDLQVSAMQHQTVLELTETGVEAAAASAISVARTLLVFEVQQPFLFVLWDQQHKFPVFMGRVYDPRA

SEQ ID NO: 4 - protein, 478 aa, synthetic construct
  REGION 1..478: note = Synthetic: SERPING1 polypeptide
NPNATSSSSQDPESLQDRGEGKVATTVISKMLFVEPILEVSSLPTTNSTTNSATKITANTTDEPTTQPTTEPTTQPTIQPTQPTTQLPTDSPTQPTTGSFCPGPVTLCSDLESHSTEAVLGDALVDFSLKLYHAFSAMKKVETNMAFSPFSIASLLTQVLLGAGENTKTNLESILSYPKDFTCVHQALKGFTTKGVTSVSQIFHSPDLAIRDTFVNASRTLYSSSPRVLSNNSDANLELINTWVAKNTNNKISRLLDSLPSDTRLVLLNAIYLSAKWKTTFDPKKTRMEPFHFKNSVIKVPMMNSKKYPVAHFIDQTLKAKVGQLQLSHNLSLVILVPQNLKHRLEDMEQALSPSVFKAIMEKLEMSKFQPTLLTLPRIKVTTSQDMLSIMEKLEFFDFSYDLNLCGLTEDPDLQVSAMQHQTVLELTETGVEAAAASAISVARTLLVFEVQQPFLFVLWDQQHKFPVFMGRVYDPRA

SEQ ID NO: 5 - protein, 478 aa, synthetic construct
  REGION 1..478: note = Synthetic: VTN polypeptide
MAPLRPLLILALLAWVALADQESCKGRCTEGFNVDKKCQCDELCSYYQSCCTDYTAECKPQVTRGDVFTMPEDEYTVYDDGEEKNNATVHEQVGGPSLTSDLQAQSKGNPEQTPVLKPEEEAPAPEVGASKPEGIDSRPETLHPGRPQPPAEEELCSGKPFDAFTDLKNGSLFAFRGQYCYELDEKAVRPGYPKLIRDVWGIEGPIDAAFTRINCQGKTYLFKGSQYWRFEDGVLDPDYPRNISDGFDGIPDNVDAALALPAHSYSGRERVYFFKGKQYWEYQFQHQPSQEECEGSSLSAVFEHFAMMQRDSWEDIFELLFWGRTSAGTRQPQFISRDWHGVPGQVDAAMAGRIYISGMAPRPSLAKKQRFRHRNRKGYRSQRGHSRGRNQNSRRPSRATWLSLFSSEESNLGANNYDDYRMDWLVPATCEPIQSVFFFSGDKYYRVNLRTRRVDTVDPPYPRSIAQYWLGCPAPGHL

SEQ ID NO: 6 - protein, 459 aa, synthetic construct
  REGION 1..459: note = Synthetic: VTN polypeptide
DQESCKGRCTEGFNVDKKCQCDELCSYYQSCCTDYTAECKPQVTRGDVFTMPEDEYTVYDDGEEKNNATVHEQVGGPSLTSDLQAQSKGNPEQTPVLKPEEEAPAPEVGASKPEGIDSRPETLHPGRPQPPAEEELCSGKPFDAFTDLKNGSLFAFRGQYCYELDEKAVRPGYPKLIRDVWGIEGPIDAAFTRINCQGKTYLFKGSQYWRFEDGVLDPDYPRNISDGFDGIPDNVDAALALPAHSYSGRERVYFFKGKQYWEYQFQHQPSQEECEGSSLSAVFEHFAMMQRDSWEDIFELLFWGRTSAGTRQPQFISRDWHGVPGQVDAAMAGRIYISGMAPRPSLAKKQRFRHRNRKGYRSQRGHSRGRNQNSRRPSRATWLSLFSSEESNLGANNYDDYRMDWLVPATCEPIQSVFFFSGDKYYRVNLRTRRVDTVDPPYPRSIAQYWLGCPAPGHL

SEQ ID NO: 7 - protein, 379 aa, synthetic construct
  REGION 1..379: note = Synthetic: VTN polypeptide
DQESCKGRCTEGFNVDKKCQCDELCSYYQSCCTDYTAECKPQVTRGDVFTMPEDEYTVYDDGEEKNNATVHEQVGGPSLTSDLQAQSKGNPEQTPVLKPEEEAPAPEVGASKPEGIDSRPETLHPGRPQPPAEEELCSGKPFDAFTDLKNGSLFAFRGQYCYELDEKAVRPGYPKLIRDVWGIEGPIDAAFTRINCQGKTYLFKGSQYWRFEDGVLDPDYPRNISDGFDGIPDNVDAALALPAHSYSGRERVYFFKGKQYWEYQFQHQPSQEECEGSSLSAVFEHFAMMQRDSWEDIFELLFWGRTSAGTRQPQFISRDWHGVPGQVDAAMAGRIYISGMAPRPSLAKKQRFRHRNRKGYRSQRGHSRGRNQNSRRPSR

SEQ ID NO: 8 - protein, 80 aa, synthetic construct
  REGION 1..80: note = Synthetic: VTN polypeptide
ATWLSLFSSEESNLGANNYDDYRMDWLVPATCEPIQSVFFFSGDKYYRVNLRTRRVDTVDPPYPRSIAQYWLGCPAPGHL

SEQ ID NO: 9 - protein, 282 aa, synthetic construct
  REGION 1..282: note = Synthetic: C1QBP polypeptide
MLPLLRCVPRVLGSSVAGLRAAAPASPFRQLLQPAPRLCTRPFGLLSVRAGSERRPGLLRPRGPCACGCGCGSLHTDGDKAFVDFLSDEIKEERKIQKHKTLPKMSGGWELELNGTEAKLVRKVAGEKITVTFNINNSIPPTFDGEEEPSQGQKVEEQEPELTSTPNFVVEVIKNDDGKKALVLDCHYPEDEVGQEDEAESDIFSIREVSFQSTGESEWKDTNYTLNTDSLDWALYDHLMDFLADRGVDNTFADELVELSTALEHQEYITFLEDLKSFVKSQ

SEQ ID NO: 10 - protein, 209 aa, synthetic construct
  REGION 1..209: note = Synthetic: C1QBP polypeptide
LHTDGDKAFVDFLSDEIKEERKIQKHKTLPKMSGGWELELNGTEAKLVRKVAGEKITVTFNINNSIPPTFDGEEEPSQGQKVEEQEPELTSTPNFVVEVIKNDDGKKALVLDCHYPEDEVGQEDEAESDIFSIREVSFQSTGESEWKDTNYTLNTDSLDWALYDHLMDFLADRGVDNTFADELVELSTALEHQEYITFLEDLKSFVKSQ

SEQ ID NO: 11 - protein, 207 aa, synthetic construct
  REGION 1..207: note = Synthetic: TIMP1 polypeptide
MAPFEPLASGILLLLWLIAPSRACTCVPPHPQTAFCNSDLVIRAKFVGTPEVNQTTLYQRYEIKMTKMYKGFQALGDAADIRFVYTPAMESVCGYFHRSHNRSEEFLIAGKLQDGLLHITTCSFVAPWNSLSLAQRRGFTKTYTVGCEECTVFPCLSIPCKLQSGTHCLWTDQLLQGSEKGFQSRHLACLPREPGLCTWQSLRSQIA

SEQ ID NO: 12 - protein, 220 aa, synthetic construct
  REGION 1..220: note = Synthetic: TIMP2 polypeptide
MGAAARTLRLALGLLLLATLLRPADACSCSPVHPQQAFCNADVVIRAKAVSEKEVDSGNDIYGNPIKRIQYEIKQIKMFKGPEKDIEFIYTAPSSAVCGVSLDVGGKKEYLIAGKAEGDGKMHITLCDFIVPWDTLSTTQKKSLNHRYQMGCECKITRCPMIPCYISSPDECLWMDWVTEKNINGHQAKFFACIKRSDGSCAWYRGAAPPKQEFLDIEDP

SEQ ID NO: 13 - protein, 1074 aa, synthetic construct
  REGION 1..1074: note = Synthetic: SEMA5A polypeptide
MKGTCVIAWLFSSLGLWRLAHPEAQGTTQCQRTEHPVISYKEIGPWLREFRAKNAVDFSQLTFDPGQKELVVGARNYLFRLQLEDLSLIQAVEWECDEATKKACYSKGKSKEECQNYIRVLLVGGDRLFTCGTNAFTPVCTNRSLSNLTEIHDQISGMARCPYSPQHNSTALLTAGGELYAATAMDFPGRDPAIYRSLGILPPLRTAQYNSKWLNEPNFVSSYDIGNFTYFFFRENAVEHDCGKTVFSRAARVCKNDIGGRFLLEDTWTTFMKARLNCSRPGEVPFYYNELQSTFFLPELDLIYGIFTTNVNSIAASAVCVFNLSAIAQAFSGPFKYQENSRSAWLPYPNPNPHFQCGTVDQGLYVNLTERNLQDAQKFILMHEVVQPVTTVPSFMEDNSRFSHVAVDVVQGREALVHIIYLATDYGTIKKVRVPLNQTSSSCLLEEIELFPERRREPIRSLQILHSQSVLFVGLREHVVKIPLKRCQFYRTRSTCIGAQDPYCGWDVVMKKCTSLEESLSMTQWEQSISACPTRNLTVDGHFGVWSPWTPCTHTDGSAVGSCLCRTRSCDSPAPQCGGWQCEGPGMEIANCSRNGGWTPWTSWSPCSTTCGIGFQVRQRSCSNPTPRHGGRVCVGQNREERYCNEHLLCPPHMFWTGWGPWERCTAQCGGGIQARRRICENGPDCAGCNVEYQSCNTNPCPELKKTTPWTPWTPVNISDNGGHYEQRFRYTCKARLADPNLLEVGRQRIEMRYCSSDGTSGCSTDGLSGDFLRAGRYSAHTVNGAWSAWTSWSQCSRDCSRGIRNRKRVCNNPEPKYGGMPCLGPSLEYQECNILPCPVDGVWSCWSPWTKCSATCGGGHYMRTRSCSNPAPAYGGDICLGLHTEEALCNTQPCPESWSEWSDWSECEASGVQVRARQCILLFPMGSQCSGNTTESRPCVFDSNFIPEVSVARSSSVEEKRCGEFNMFHMIAVGLSSSILGCLLTLLVYTYCQRYQQQSHDATVIHPVSPAPLNTSITNHINKLDKYDSVEAIKAFNKNNLILEERNKYFNPHLTGKTYSNAYFTDLNNYDEY

SEQ ID NO: 14 - protein, 1461 aa, synthetic construct
  REGION 1..1461: note = Synthetic: NEO1 polypeptide
MAAERGARRLLSTPSFWLYCLLLLGRRAPGAAAARSGSAPQSPGASIRTFTPFYFLVEPVDTLSVRGSSVILNCSAYSEPSPKIEWKKDGTFLNLVSDDRRQLLPDGSLFISNVVHSKHNKPDEGYYQCVATVESLGTIISRTAKLIVAGLPRFTSQPEPSSVYAGNNAILNCEVNADLVPFVRWEQNRQPLLLDDRVIKLPSGMLVISNATEGDGGLYRCVVESGGPPKYSDEVELKVLPDPEVISDLVFLKQPSPLVRVIGQDVVLPCVASGLPTPTIKWMKNEEALDTESSERLVLLAGGSLEISDVTEDDAGTYFCIADNGNETIEAQAELTVQAQPEFLKQPTNIYAHESMDIVFECEVTGKPTPTVKWVKNGDMVIPSDYFKIVKEHNLQVLGLVKSDEGFYQCIAENDVGNAQAGAQLIILEHAPATTGPLPSAPRDVVASLVSTRFIKLTWRTPASDPHGDNLTYSVFYTKEGIARERVENTSHPGEMQVTIQNLMPATVYIFRVMAQNKHGSGESSAPLRVETQPEVQLPGPAPNLRAYAASPTSITVTWETPVSGNGEIQNYKLYYMEKGTDKEQDVDVSSHSYTINGLKKYTEYSFRVVAYNKHGPGVSTPDVAVRTLSDVPSAAPQNLSLEVRNSKSIMIHWQPPAPATQNGQITGYKIRYRKASRKSDVTETLVSGTQLSQLIEGLDRGTEYNFRVAALTINGTGPATDWLSAETFESDLDETRVPEVPSSLHVRPLVTSIVVSWTPPENQNIVVRGYAIGYGIGSPHAQTIKVDYKQRYYTIENLDPSSHYVITLKAFNNVGEGIPLYESAVTRPHTDTSEVDLFVINAPYTPVPDPTPMMPPVGVQASILSHDTIRITWADNSLPKHQKITDSRYYTVRWKTNIPANTKYKNANATTLSYLVTGLKPNTLYEFSVMVTKGRRSSTWSMTAHGTTFELVPTSPPKDVTVVSKEGKPKTIIVNWQPPSEANGKITGYIIYYSTDVNAEIHDWVIEPVVGNRLTHQIQELTLDTPYYFKIQARNSKGMGPMSEAVQFRTPKADSSDKMPNDQASGSGGKGSRLPDLGSDYKPPMSGSNSPHGSPTSPLDSNMLLVIIVSVGVITIVVVVIIAVFCTRRTTSHQKKKRAACKSVNGSHKYKGNSKDVKPPDLWIHHERLELKPIDKSPDPNPIMTDTPIPRNSQDITPVDNSMDSNIHQRRNSYRGHESEDSMSTLAGRRGMRPKMMMPFDSQPPQPVISAHPIHSLDNPHHHFHSSSLASPARSHLYHPGSPWPIGTSMSLSDRANSTESVRNTPSTDTMPASSSQTCCTDHQDPEGATSSSYLASSQEEDSGQSLPTAHVRPSHPLKSFAVPAIPPPGPPTYDPALPSTPLLSQQALNHHIHSVKTASIGTLGRSRPPMPVVVPSAPEVQETTRMLEDSESSYEPDELTKEMAHLEGLMKDLNAITTA

SEQ ID NO: 15 - protein, 770 aa, synthetic construct
  REGION 1..770: note = Synthetic: APP polypeptide
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

(19) United States
(12) Patent Application Publication
(10) Pub. No.: US 2025/0009807 A1
(43) Pub. Date: Jan. 9, 2025

(54) COMPOSITIONS AND METHODS OF TREATING CORNEAL SCARRING

(71) Applicant: UNIVERSITY OF PITTSBURGH - OF THE COMMONWEALTH SYSTEM OF HIGHER EDUCATION, Pittsburgh, PA (US)

(72) Inventors: Yiqin DU, Pittsburgh, PA (US); Ajay KUMAR, Pittsburgh, PA (US)

(73) Assignee: UNIVERSITY OF PITTSBURGH - OF THE COMMONWEALTH SYSTEM OF HIGHER EDUCATION, Pittsburgh, PA (US)

(21) Appl. No.: 18/892,287

(22) Filed: Sep. 20, 2024

Related U.S. Application Data
(63) Continuation of application No. PCT/US2023/023419, filed on May 24, 2023.
(60) Provisional application No. 63/345,386, filed on May 24, 2022.

(51) Int. Cl. A61K 35/30

(52) U.S. Cl. CPC A61K 35/30

(57) ABSTRACT

The present disclosure relates to compositions and methods for preventing and/or treating corneal scarring. The present disclosure further provides kits for performing such methods.

### CROSS-REFERENCE TO RELATED APPLICATIONS

This application is a continuation of International Application No. PCT/US2023/023419 filed on May 24, 2023, which claims priority to U.S. Provisional Patent Application Ser. No. 63/345,386 filed on May 24, 2022, both of which are hereby incorporated by reference in their entireties.

### GOVERNMENT LICENSE RIGHTS

This invention was made with government support under grant numbers EY008098, EY025643, and EY024642 awarded by the National Institutes of Health (NIH). The government has certain rights in the invention.

### SEQUENCE LISTING

The instant application contains a Sequence Listing, which has been submitted in XML format via EFS-Web and is hereby incorporated by reference in its entirety. Said XML copy, created on Sep. 12, 2024, is named 072396.1037_seq.xml and is 26,565 bytes in size.

### FIELD OF INVENTION

The present disclosure relates to the use of novel pharmaceutical compositions to prevent and/or treat corneal scarring.

### BACKGROUND

Corneal stroma injuries normally result in permanent haze and scarring, therefore impairing vision. As of now, the best treatment for corneal scarring with reduced vision is corneal transplantation, which requires donor tissues and includes the risk of graft rejection. Although corneal transplantation is the most successful organ transplantation in the human body, long-term success is a challenge as survival rates reduced from 89% at 5 years to 17% at 23 years (Kelly et al., Arch Ophthalmol 129, 691-697 (2011)). Currently, there are 12.7 million people on the waiting list for corneal transplantation worldwide (Gain et al., JAMA Ophthalmol 134, 167-173 (2016)). Accordingly, there remains a need in the art for novel therapies in the treatment of corneal scarring.

### SUMMARY

The present disclosure provides compositions and methods for treating corneal scarring in a subject.

In certain non-limiting embodiments, the present disclosure provides a pharmaceutical composition comprising at least one complement inhibitor protein. In certain embodiments, the complement inhibitor protein comprises a CD59 polypeptide or a functional fragment thereof. In certain embodiments, the complement inhibitor protein comprises a SERPING1 polypeptide or a functional fragment thereof. In certain embodiments, the complement inhibitor protein comprises a vitronectin (VTN) polypeptide or a functional fragment thereof. In certain embodiments, the complement inhibitor protein comprises a CIQBP polypeptide or a functional fragment thereof.

In certain non-limiting embodiments, the present disclosure also provides a pharmaceutical composition comprising a CD59 polypeptide or a functional fragment thereof, a SERPING1 polypeptide or a functional fragment thereof, a VTN polypeptide or a functional fragment thereof, a CIQBP polypeptide or a functional fragment thereof, or a combination thereof.

In certain embodiments, the CD59 polypeptide comprises an amino acid sequence at least 80% identical to the amino acid sequence set forth in SEQ ID NO: 1 or SEQ ID NO: 2. In certain embodiments, the CD59 polypeptide comprises the amino acid sequence set forth in

SEQ ID NO: 1 or SEQ ID NO: 2. In certain embodiments, the SERPING1 polypeptide comprises an amino acid sequence at least 80% identical to the amino acid sequence set forth in SEQ ID NO: 3 or SEQ ID NO: 4. In certain embodiments, the SERPING1 polypeptide comprises the amino acid sequence set forth in SEQ ID NO: 3 or SEQ ID NO: 4. In certain embodiments, the VTN polypeptide comprises an amino acid sequence at least 80% identical to the amino acid sequence set forth in SEQ ID NO: 5, SEQ ID NO: 6, SEQ ID NO: 7, or SEQ ID NO: 8. In certain embodiments, the VTN polypeptide comprises the amino acid sequence set forth in SEQ ID NO: 5, SEQ ID NO: 6, SEQ ID NO: 7, or SEQ ID NO: 8. In certain embodiments, the C1QBP polypeptide comprises an amino acid sequence at least 80% identical to the amino acid sequence set forth in SEQ ID NO: 9 or SEQ ID NO: 10. In certain embodiments, the C1QBP polypeptide comprises the amino acid sequence set forth in SEQ ID NO: 9 or SEQ ID NO: 10.

In certain embodiments, the pharmaceutical composition disclosed herein further comprises at least one polypeptide selected from the group consisting of TIMP1, TIMP2,SEMA5A, FKBP4, VCL, LGALS1, CTSZ, PQBP1, TGFB1, LAMB1, PRPF19, LEPRE1, LRP1, VASP, YWHAH, SERPINF1, JAG1, GPC1, YWHAE, IQGAP1, ARHGDIA, SOD1, THY1, APP, DBN1, MAP1B, AXL, TWF2, COL3A1, ALCAM, YWHAG, DPYSL2, CALR, CSF1, FLRT2, CAPRIN1, NEO1, EPB41L3, DPYSL3, APOD, HMGB1, VAPA, CRKL, FN1, PDLIM7, SPTBN1, MAP4, ENAH, EZR, PTPRK, FLNA, HSP90AB1, SPTAN1, FAM129B, CTTN, ACTR2, EXT1, POSTN, XRCC5, SPOCK1, ITGB1, TRIOBP, LAMB2, RHOA, PRMT1, STMN1, SERPINE2, EIF4G1, DBNL, PTK7, WDR1, CFL1, MANF, DAG1, VIM, B2M, LDLR, VEGFA, YAP1, VEGFC, PDLIM5, CRABP2, and SPON2.

In certain embodiments, the at least one polypeptide comprises a TIMP1 polypeptide or a functional fragment thereof. In certain embodiments, the TIMP1 polypeptide comprises an amino acid sequence at least 80% identical to the amino acid sequence set forth in SEQ ID NO: 11. In certain embodiments, the TIMP1 polypeptide comprises the amino acid sequence set forth in SEQ ID NO: 11.

In certain embodiments, the at least one polypeptide comprises a TIMP2 polypeptide or a functional fragment thereof. In certain embodiments, the TIMP2 polypeptide comprises an amino acid sequence at least 80% identical to the amino acid sequence set forth in SEQ ID NO: 12. In certain embodiments, the TIMP2 polypeptide comprises the amino acid sequence set forth in SEQ ID NO: 12.

In certain embodiments, the at least one polypeptide comprises a SEMA5A polypeptide or a functional fragment thereof. In certain embodiments, the SEMA5A polypeptide comprises an amino acid sequence at least 80% identical to the amino acid sequence set forth in SEQ ID NO: 13. In certain embodiments, the SEMA5A polypeptide comprises the amino acid sequence set forth in SEQ ID NO: 13.

In certain embodiments, the pharmaceutical composition disclosed herein further comprises at least one polypeptide selected from the group consisting of NEO1, APP, FKBP4, VCL, LGALS1, CTSZ, PQBP1, TGFB1, LAMB1, PRPF19, LEPRE1, LRP1, VASP, YWHAH, SERPINF1, JAG1, TIMP2, GPC1, YWHAE, IQGAP1, ARHGDIA, SOD1, THY1, APP, DBN1, MAP1B, AXL, TWF2, COL3A1, ALCAM, YWHAG, DPYSL2, CALR, CSF1, FLRT2, CAPRIN1, EPB41L3, DPYSL3, APOD, HMGB1, VAPA, CRKL, FN1, PDLIM7, SPTBN1, MAP4, ENAH, EZR, PTPRK, FLNA, HSP90AB1, SPTAN1, FAM129B, CTTN, ACTR2, EXT1, POSTN, SEMASA, XRCC5, SPOCK1, ITGB1, TRIOBP, LAMB2, RHOA, PRMT1, STMN1, SERPINE2, EIF4G1, DBNL, PTK7, WDR1, CFL1, MANF, DAG1, VIM, B2M, LDLR, VEGFA, YAP1, VEGFC, PDLIM5, CRABP2, and SPON2.

In certain embodiments, the at least one polypeptide comprises a NEO1 polypeptide or a functional fragment thereof. In certain embodiments, the NEO1 polypeptide comprises an amino acid sequence at least 80% identical to the amino acid sequence set forth in SEQ ID NO: 14. In certain embodiments, the NEO1 polypeptide comprises the amino acid sequence set forth in SEQ ID NO: 14.

In certain embodiments, the at least one polypeptide comprises an APP polypeptide or a functional fragment thereof. In certain embodiments, the APP polypeptide comprises an amino acid sequence at least 80% identical to the amino acid sequence set forth in SEQ ID NO: 15. In certain embodiments, the APP polypeptide comprises the amino acid sequence set forth in SEQ ID NO: 15.

In certain embodiments, the pharmaceutical composition disclosed herein further comprises at least one polypeptide selected from the group consisting of DCN, APOH, TIMP1, PLAT, TGFB1, CIQBP, COL1A1, LOX, SPARC, PDGFRA, PDGFRB, SERPING1, ANXA5, COL1A2, AXL, COL3A1, PKM, FBLN1, PEAR1, APOD, FN1, FLNA, COL5A1, GSN, ANXA1, B4GALT1, POSTN, PRKARIA, PDGFD, CORO1B, CD59, YWHAZ, CD44, CYR61, RHOA, CAPZB, SERPINE2, PTK7, DAG1, NACA, YAP1, ALDOA, PARK7, PRDX1, TMOD3, EEF2, YWHAZ, PFN1, FASN, EIF4H, TWF2, PLIN3, EIF2A, YWHAE, ENO1, RAN, FAM129B, PUF60, PKM, SPTAN1, AHNAK, FSCN1, BAG3, EPS15L1, S100A11, VAPA, SND1, AHSA1, CAPZB, CORO1B, VASP, KTN1, FLNB, ANXA2, RANBP1, DBNL, HNRNPK, PLEC, TNKS1BP1, RTN4, EEF1G, PKM, EEF1D, CAST, TAGLN2, YWHAB, CAPG, RPL29, LDHA, HSP90AB1, CALD1, CAST, SERBP1, PRDX6, CALD1, MAPRE1, NUDC, IQGAP1, CRKL, TWF1, PDLIM5, PAICS, EIF4G1, IDH1, HSPA8, PDLIM1, PCBP1, SPTBN1, RPL14, CTTN, VASN, RDX, DBN1, and DSG.

In certain non-limiting embodiments, the present disclosure further provides a pharmaceutical composition comprising a secretome harvested from a human corneal stromal stem cell (CSSC).

In certain non-limiting embodiments, the present disclosure also provides a hydrogel comprising the pharmaceutical composition disclosed herein. In certain embodiments, the hydrogel further comprises thrombin, fibrinogen, or a combination thereof.

Additionally, the present disclosure provides a contact lens comprising a pharmaceutical composition or a hydrogel disclosed herein.

In certain non-limiting embodiments, the present disclosure also provides for a kit for preventing, reducing the risk of, and/or treating corneal scarring in a subject in need thereof, comprising an effective amount of a pharmaceutical composition, a hydrogel, and/or a contact lens.

Finally, the present disclosure provides a method of preventing, reducing the risk of, and/or treating corneal scarring in a subject in need thereof. In certain embodiments, the method comprises administering an effective amount of a pharmaceutical composition, a hydrogel, or a contact lens disclosed herein. In certain embodiments, the method comprises administering an effective amount of at least one complement inhibitory protein. In certain embodiments, the method comprises administering a secretome harvested from a corneal stromal stem cell (CSSC).

### BRIEF DESCRIPTION OF THE DRAWINGS

For a more complete understanding of the present disclosure and its features and advantages, reference is now made to the following description, taken in conjunction with the accompanying drawings.

FIGS. 1A-1D illustrate the stem cell characteristics of human corneal stromal stem cells (CSSC) and cell viability post secretome harvesting. FIG. 1A shows dot plots from flow cytometry analysis of stem cell markers in CSSC (n=3). FIG. 1B shows a bar graph depicting the expression of stem cell markers in CSSC (n=3). FIG. 1C shows the Annexin V-7AAD flow cytometry analysis for cell viability assessment in corneal fibroblasts and CSSC post secretome harvesting, after incubation in serum-free media. Gate was set on unstained control cells for normalizing the background fluorescence and isotype controls for the elimination of non-specific staining. 100,000 events per tube were acquired. Results are representative of Mean±SD. FIG. 1D shows live-cell fluorescence laser scanning confocal microscopy for cell viability in corneal fibroblasts and CSSC post secretome harvesting. Calcein AM and Hoechst 33342 were used at a dilution rate of 1:1000 and 1:2000 respectively. Scale bars-100 μm.

FIGS. 2A-2E illustrate the effect of CSSC secretome (Scr) on corneal wound healing and inflammation. FIG. 2A shows optical coherence tomography (OCT) analysis of mouse corneas showing wounded corneas with different treatments (three days following treatment). FIG. 2B shows a table reporting number of corneas with complete healing, partial healing, and no healing (n=19-20). FIG. 2C shows immunofluorescent images showing expression of inflammation related antibodies CD45, F4/80, and GR1 in the cornea, scale bars−50 μm. FIG. 2D shows dot plots represented as Mean±SD for inflammation related antibodies CD45,F4/80, and GR1. Each dot on graph represents mean fluorescence intensity (MFI) from one corneal section. 6-8 corneal sections were imaged per eye (n=3 corneas). FIGS. 2A-2D correspond to analyses performed at day 3 after wound treatment. FIG. 2E shows the CSSC secretome protective effect on mouse corneas at day 7 after wound treatment. The top panel of FIG. 2E shows representative Hematoxylin & Eosin (H&E) staining images and the bottom panel shows representative OCT images.

FIGS. 3A and 3B illustrate the effect of corneal stromal stem cell secretome on inflammatory cells. FIG. 3A shows dot plots showing gating strategy for the inflammatory cells in the corneas. Cells were first excluded based on violet live/dead staining. Live CD45+ cells were further gated as CD11b+/− cells and out of those, GR1+/− and F4/80+/− cells were counted. FIG. 3B shows dot plots showing percentage positive inflammatory cells for different antibodies, CD45 (n=8−10), CD11b/F4/80 (n=8−9), and CD11b/Gr1 (n=5).

FIGS. 4A-4D illustrate the effect of corneal stromal stem cell secretome on extracellular matrix (ECM) deposition and fibrosis. FIGS. 4A and 4B show immunofluorescent images and dot plots showing expression and quantification of mean fluorescence intensity (MFI) for ECM markers Col IV and Col3A1 in the cornea. FIGS. 4C and 4D show immunofluorescence images and quantification of MFI for the fibrotic markers SPARC and α-SMA in the cornea, scale bars−50 μm. Dot plots are represented as Mean±SD. Each dot on graph represents MFI from one corneal section. 6-8 sections were photographed per eye (n=3 corneas).

FIGS. 5A and 5B illustrate proteomic analysis of corneal stromal stem cell and fibroblast secretomes. FIG. 5A shows an interactome analysis of secretome proteins from both CSSC and fibroblasts depicting interacting proteins for wound healing, more in CSSC and fewer in corneal fibroblasts, as analyzed by String v11, (n=2 each). FIG. 5B shows a heatmap depicting hierarchical clustering and the differential expression of cell-adhesion proteins between CSSC and corneal fibroblast secretome. Heatmaps were generated using R package heatmap (version 1.0.12).

FIGS. 6A-6E illustrate corneal stromal stem cell secretome inducing sensory nerve regeneration by key proteins. FIG. 6A shows a schematic representing the wholemount cornea and the scheme for how corneal neurons were quantified for the mean fluorescence intensity of β-3 tubulin (image adapted from Birender.com, left panel). Immunofluorescent stitched images composed of multiple z-stacks acquired for the expression of neuronal marker β-3 tubulin in wholemount of corneas (middle panel). Corneal nerve plexuses are visible in control and CSSC secretome treated areas whereas almost lost in sham. Dot plots (right panel) showing quantification of mean fluorescence intensity (MFI) of β-3 tubulin staining. FIG. 6B shows a schematic representing wholemount cornea and how mean fluorescence intensity (MFI) was quantified for p-substance (image adapted from Birender.com, left panel), confocal images showing stitched z-stacks for expression of P-substance in wholemount corneas (middle panel), Dot plots (right panel) showing quantification of mean fluorescence intensity (MFI) of P-substance staining. One dot on the bar graph represents quantification from one ribbon of the cornea (n=3 corneas). Data is represented as Mean±SD. FIGS. 6A-6B correspond to analyses performed at day 3 after wound treatment. FIG. 6C shows representative β-3 tubulin staining images on the corneal wholemounts demonstrating the CSSC Scr effect to preserve the corneal sensory nerve axons at day 7 after wound treatment. The axons extend to the central corneas with CSSC Scr treatment, similar to that of naïve controls, while the axons didn't extend to the central region in the corneas treated with Fibro Scr and Sham. FIGS. 6D and 6E show an interactome analysis of secretome proteins from both CSSC and fibroblasts depicting interaction between proteins involved in the generation of neurons in both CSSC and fibroblasts and axon guidance in CSSC, as analyzed by String v11, (n=2 each).

FIGS. 7A-7C illustrate proteomic characterization of secretome proteins involved in neuron differentiation and projection development. FIG. 7A shows interactome analysis of secretome proteins from CSSC and corneal fibroblasts depicting higher number of proteins involved in regulation of neuron differentiation as compared to corneal fibroblasts. FIG. 7B shows an interactome showing a similar pattern for proteins involved in the development of neuron projections, as analyzed by String v11, (n=2 each). FIG. 7C shows a schematic figure depicting an overall scheme for neuroprotective effect of CSSC secretome on corneal wound healing and sensory nerve regeneration (adapted from Biorender.com).

FIGS. 8A and 8B illustrate corneal cell death analysis using TUNEL staining. FIG. 8A shows immunofluorescent images showing TUNEL+ cells in the cryosections of central and peripheral corneas. The higher number of TUNEL+ cells are clearly visible in sham and fibroblast secretome treated corneas, scale bars−50 μm. FIG. 8B shows dot plots showing quantification of mean TUNEL+ cells in the cornea. Data is represented as Mean±SD. Each dot on graph represents counts from one corneal section. 3-4 sections were photographed per eye (n=3 corncas).

FIGS. 9A and 9D illustrate corneal stromal stem cell secretome inhibiting complements. FIG. 9A shows interactome analysis for secretome proteins from both CSSC and fibroblasts showing presence of four unique proteins CD59, Vitronectin (VTN), SERPING1, and CIQBP in the CSSC secretome as analyzed by String v11, (n=2 each). FIG. 9B shows immunofluorescent images showing autoantibody staining using a goat anti-mouse IgG antibody and dot plots showing mean fluorescence intensity (MFI) quantification showing a higher formation of autoantibodies in sham and fibroblast secretome treated corneas. FIG. 9C shows immunofluorescent images showing increased expression of vitronectin and CD59 in the control and CSSC secretome treated corneas while sham and fibroblast secretome treated corneas displayed reduced expression. FIG. 9D shows dot plots represent quantification of the MFI for CD59 and vitronectin. Scale bars−50 μm. Data is represented as Mean±SD. Dots on graph represent MFI from one corneal section. 6-8 sections were photographed per eye (n=3 corneas).

FIG. 10 illustrates a proposed model of corneal regeneration by CSSC secretome.

### DETAILED DESCRIPTION

The present disclosure relates to composition and methods to prevent and/or treat corneal scarring. The present disclosure is based, in part, on the discovery that certain components of the corneal stromal stem cells (CSSC) secretome can promote wound scarless wound healing and nerve regeneration.

For purposes of clarity of disclosure and not by way of limitation, the detailed description is divided into the following subsections:
1. Definitions;
2. Pharmaceutical Compositions;
3. Contact Lenses;
4. Methods of Treatment; and
5. Kits.

### 1. Definitions

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the disclosure and how to make and use them.

As used herein, the use of the word “a” or “an” when used in conjunction with the term “comprising” in the claims and/or the specification can mean “one,” but it is also consistent with the meaning of “one or more,” “at least one,” and “one or more than one.”

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The present disclosure also contemplates other embodiments “comprising,” “consisting of”, and “consisting essentially of,” the embodiments or elements presented herein, whether explicitly set forth or not.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a valuc.

An “individual” or “subject” herein is a vertebrate, such as a human or non-human animal, for example, a mammal. Mammals include, but are not limited to, humans, non-human primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

As used herein, the term “disease” refers to any condition or disorder that damages or interferes with the normal function of a cell, tissue, or organ.

An “effective amount” or “therapeutically effective amount” is an amount effective, at dosages and for periods of time necessary, that produces a desired effect, e.g., the desired therapeutic or prophylactic result. In certain embodiments, an effective amount can be formulated and/or administered in a single dose. In certain embodiments, an effective amount can be formulated and/or administered in a plurality of doses, for example, as part of a dosing regimen.

“Inhibitors” or “antagonists,” as used herein, refer to modulating compounds that reduce, decrease, block, prevent, delay activation, inactivate, desensitize, or down-regulate the biological activity and/or expression of a receptor or pathway of interest. The term “antagonist” includes full, partial, and neutral antagonists as well as inverse agonists.

As used herein, the term “antagonist” refers to modulating compounds that reduce, decrease, block, prevent, delay activation, inactivate, desensitize or down-regulate the biological activity and/or expression of a receptor or pathway of interest. In certain embodiments, the term “antagonist” includes full, partial, and neutral antagonists as well as inverse agonists.

As used herein, the term “agonist” refers to modulating compounds that increase, induce, stimulate, open, activate, facilitate, enhance activation, sensitize or upregulate a receptor or pathway of interest. In certain embodiments, the term “agonist” includes full and partial agonists.

The term “nucleic acid molecule” and “nucleotide sequence,” as used herein, refers to a single or double-stranded covalently-linked sequence of nucleotides in which the 3′ and 5′ ends on each nucleotide are joined by phosphodiester bonds. The nucleic acid molecule can include deoxyribonucleotide bases or ribonucleotide bases, and can be manufactured synthetically in vitro or isolated from natural sources.

The terms “polypeptide,” “peptide,” “amino acid sequence” and “protein,” used interchangeably herein, refer to a molecule formed from the linking of at least two amino acids. The link between one amino acid residue and the next is an amide bond and is sometimes referred to as a peptide bond. A polypeptide can be obtained by a suitable method known in the art, including isolation from natural sources, expression in a recombinant expression system, chemical synthesis, or enzymatic synthesis. The terms can apply to amino acid polymers in which one or more amino acid residues is an artificial chemical mimetic of a corresponding naturally occurring amino acid, as well as to naturally occurring amino acid polymers and non-naturally occurring amino acid polymers. In certain embodiments, the polypeptide can have one or more conservative amino acid substitutions. As used herein, “conservative amino acid substitution(s)” are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. Amino acids can also be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. In certain embodiments, no more than one, no more than two, no more than three, no more than four, or no more than five residues within a specified sequence are altered. Exemplary conservative amino acid substitutions are shown in Table 1 below.

TABLE 1

| Original Residue | Exemplary Conservative Amino Acid Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gin (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe |
| Leu (L) | Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala |

As used herein, the percent homology between two amino acid sequences is equivalent to the percent identity between the two sequences. The percent identity between the two sequences is a function of the number of identical positions shared by the sequences (i.e., % homology= #of identical positions/total #of positions×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

As used herein, the term “disease” refers to any condition or disorder that damages or interferes with the normal function of a cell, tissue, or organ.

As used herein, the term “treating” or “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing aneurysms, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment can prevent the onset of the disorder or a symptom of the disorder in a subject at risk for the disorder or suspected of having the disorder.

As used herein, “a functional fragment” of a molecule or polypeptide includes a fragment of the molecule or polypeptide that retains at least about 80%, at least about 85%, at least about 90%, at least about 95%, or at least about 100% of the primary function of the molecule or polypeptide.

### 2. Pharmaceutical Compositions

The present disclosure provides pharmaceutical compositions for use in the methods disclosed herein. In certain embodiments, the pharmaceutical compositions disclosed herein comprise a polypeptide that promotes wound healing. In certain embodiments, the pharmaceutical compositions disclosed herein comprise a polypeptide that promotes nerve regeneration. In certain embodiments, the pharmaceutical compositions disclosed herein comprise a polypeptide that inhibits the complement system. In certain embodiments, the pharmaceutical compositions disclosed herein can reversibly or irreversibly inhibit the complement system resulting in the healing of a corneal wound.

In certain embodiments, the pharmaceutical composition comprises a complement inhibitory protein. As used herein, the term “complement inhibitory protein” refers to a protein, a polypeptide, or a fragment thereof that can target different levels and steps of the complement cascade to thereby inhibiting the same.

Complement is known to be the first defense against non-self-cells or unwanted host clements. The spectrum of complement-mediated functions ranges from direct cell lysis to the control of humoral and adaptive immunity. The complement system also regulates several immunological and inflammatory processes that contribute to body homeostasis. Physiologically, there are three pathways of complement activation: the classical, the alternative, and the lectin pathways. While these three complement pathways differ in their mechanisms of target recognition, they converge in the activation of the central component C3.

After this activation, C5 is cleaved, and the assembly of the pore-like membrane attack complex (MAC) is initiated. The enzymatic cleavage of C3 and C5 leads to the production and release of anaphylatoxins C3a and C5a, two important inflammatory mediators and chemoattractants. Additional information on complement physiology can be found in Murphy and Casey, Janeway's immunobiology, Garland science, 2016.

In certain embodiments, the complement inhibitory protein is a CD59 polypeptide or a functional fragment thereof. In certain embodiments, the CD59 polypeptide is a human CD59 polypeptide. CD59 is a glycoprotein, also known as membrane attack complex inhibitory protein (MAC-IP), membrane inhibitor of reactive lysis (MIRL), or protectin, that belongs to the LY6/uPAR/alpha-neurotoxin protein family. Physiologically, CD59 inhibits the formation of MAC pores in the membranes of expressing cells. CD59 is also described as a “suicide inhibitor” because it locks onto C8 in the forming MAC and blocks the recruitment of C9 into the complex. CD59 binds complement C8 and/or C9 during MAC formation, thus preventing cells from generating autoantibodies, which cause cell death, kill themselves, and inhibiting the terminal pathway of complement cascade.

In certain embodiments, the CD59 polypeptide comprises an amino acid sequence at about 80% identical to the amino acid sequence set forth in SEQ ID NO: 1. In certain embodiments, the CD59 polypeptide comprises an amino acid sequence at about 85%, at least about 90%, at least about 95%, at least about 96%, at least about 97%, at least about 98%, or at least about 99% identical to the amino acid sequence set forth in SEQ ID NO: 1. In certain embodiments, the CD59 polypeptide comprises the amino acid sequence set forth in SEQ ID NO: 1. In certain embodiments, the CD59 polypeptide consists of the amino acid sequence set forth in SEQ ID NO: 1. SEQ ID NO. 1 is provided below.

[SEQ ID NO: 1]
MGIQGGSVLFGLLLVLAVFCHSGHSLQCYNCPNPTADCKTAVNCSSDFDA
CLITKAGLQVYNKCWKFEHCNFNDVTTRLRENELTYYCCKKDLCNFNEQL
ENGGTSLSEKTVLLLVTPFLAAAWSLHP

In certain embodiments, the CD59 polypeptide comprises a signal peptide. In certain embodiments, the CD59 polypeptide lacks a signal peptide. In certain embodiments, the CD59 polypeptide comprises a propeptide. In certain embodiments, the CD59 polypeptide lacks a propeptide.

In certain embodiments, the CD59 polypeptide comprises an amino acid sequence at 80% identical to the amino acid sequence set forth in SEQ ID NO: 2. In certain embodiments, the CD59 polypeptide comprises an amino acid sequence at about 85%, at least about 90%, at least about 95%, at least about 96%, at least about 97%, at least about 98%, or at least about 99% identical to the amino acid sequence set forth in SEQ ID NO: 2. In certain embodiments, the CD59 polypeptide comprises the amino acid sequence set forth in SEQ ID NO: 2. In certain embodiments, the CD59 polypeptide consists of the amino acid sequence set forth in SEQ ID NO: 2. SEQ ID NO. 2 is provided below.

[SEQ ID NO: 2]
LQCYNCPNPTADCKTAVNCSSDFDACLITKAGLQVYNKCWKFEHCNFNDV
TTRLRENELTYYCCKKDLCNFNEQLEN

In certain embodiments, the complement inhibitory protein is a SERPING1 polypeptide or a functional fragment thereof. In certain embodiments, the SERPING1 polypeptide is a human SERPING1 polypeptide. SERPING1, also known as C1-inhibitor (C1-inh, C1 esterase inhibitor), is a protease inhibitor belonging to the serpin superfamily. Its main function is the inhibition of the complement system to prevent spontaneous activation but also as the major regulator of the contact system. SERPING1 is an efficient inhibitor of FXIIa, chymotrypsin, and kallikrein.

In certain embodiments, the SERPING1 polypeptide comprises an amino acid sequence at 80% identical to the amino acid sequence set forth in SEQ ID NO: 3. In certain embodiments, the SERPING1 polypeptide comprises an amino acid sequence at about 85%, at least about 90%, at least about 95%, at least about 96%, at least about 97%, at least about 98%, or at least about 99% identical to the amino acid sequence set forth in SEQ ID NO: 3.

In certain embodiments, the SERPING1 polypeptide comprises the amino acid sequence set forth in SEQ ID NO: 3. In certain embodiments, the SERPING1 polypeptide consists of the amino acid sequence set forth in SEQ ID NO: 3. SEQ ID NO. 3 is provided below.

[SEQ ID NO: 3]
MASRLTLLTLLLLLLAGDRASSNPNATSSSSQDPESLQDRGEGKVATTVI
SKMLFVEPILEVSSLPTTNSTTNSATKITANTTDEPTTQPTTEPTTQPTI
QPTQPTTQLPTDSPTQPTTGSFCPGPVTLCSDLESHSTEAVLGDALVDFS
LKLYHAFSAMKKVETNMAFSPFSIASLLTQVLLGAGENTKTNLESILSYP
KDFTCVHQALKGFTTKGVTSVSQIFHSPDLAIRDTFVNASRTLYSSSPRV
LSNNSDANLELINTWVAKNTNNKISRLLDSLPSDTRLVLLNAIYLSAKWK
TTFDPKKTRMEPFHFKNSVIKVPMMNSKKYPVAHFIDQTLKAKVGQLQLS
HNLSLVILVPQNLKHRLEDMEQALSPSVFKAIMEKLEMSKFQPTLLTLPR
IKVTTSQDMLSIMEKLEFFDFSYDLNLCGLTEDPDLQVSAMQHQTVLELT
ETGVEAAAASAISVARTLLVFEVQQPFLFVLWDQQHKFPVFMGRVYDPRA

In certain embodiments, the SERPING1 polypeptide comprises a signal peptide. In certain embodiments, the SERPING1 polypeptide lacks a signal peptide. In certain embodiments, the SERPING1 polypeptide comprises an amino acid sequence at 80% identical to the amino acid sequence set forth in SEQ ID NO: 4. In certain embodiments, the SERPING1 polypeptide comprises an amino acid sequence at about 85%, at least about 90%, at least about 95%, at least about 96%, at least about 97%, at least about 98%, or at least about 99% identical to the amino acid sequence set forth in SEQ ID NO: 4. In certain embodiments, the SERPING1 polypeptide comprises the amino acid sequence set forth in SEQ ID NO: 4. In certain embodiments, the SERPING1 polypeptide consists of the amino acid sequence set forth in SEQ ID NO: 4. SEQ ID NO. 4 is provided below.

[SEQ ID NO: 4]
NPNATSSSSQDPESLQDRGEGKVATTVISKMLFVEPILEVSSLPTTNSTT
NSATKITANTTDEPTTQPTTEPTTQPTIQPTQPTTQLPTDSPTQPTTGSF
CPGPVTLCSDLESHSTEAVLGDALVDFSLKLYHAFSAMKKVETNMAFSPF
SIASLLTQVLLGAGENTKTNLESILSYPKDFTCVHQALKGFTTKGVTSVS
QIFHSPDLAIRDTFVNASRTLYSSSPRVLSNNSDANLELINTWVAKNTNN
KISRLLDSLPSDTRLVLLNAIYLSAKWKTTFDPKKTRMEPFHFKNSVIKV
PMMNSKKYPVAHFIDQTLKAKVGQLQLSHNLSLVILVPQNLKHRLEDMEQ
ALSPSVFKAIMEKLEMSKFQPTLLTLPRIKVTTSQDMLSIMEKLEFFDFS
YDLNLCGLTEDPDLQVSAMQHQTVLELTETGVEAAAASAISVARTLLVFE
VQQPFLFVLWDQQHKFPVFMGRVYDPRA

In certain embodiments, the complement inhibitory protein is a vitronectin (VTN) polypeptide or a functional fragment thereof. In certain embodiments, the VTN polypeptide is a human VTN polypeptide. Vitronectin (VTN) is a multifunctional glycoprotein of 75 kD that binds to various biological ligands and plays a key role in tissue remodeling by regulating cell adhesion through binding to different types of integrins, for example via the RGD sequence.

In certain embodiments, the VTN polypeptide comprises an amino acid sequence at 80% identical to the amino acid sequence set forth in SEQ ID NO: 5. In certain embodiments, the VTN polypeptide comprises an amino acid sequence at about 85%, at least about 90%, at least about 95%, at least about 96%, at least about 97%, at least about 98%, or at least about 99% identical to the amino acid sequence set forth in SEQ ID NO: 5. In certain embodiments, the VTN polypeptide comprises the amino acid sequence set forth in SEQ ID NO: 5. In certain embodiments, the VTN polypeptide consists of the amino acid sequence set forth in SEQ ID NO: 5. SEQ ID NO. 5 is provided below.

[SEQ ID NO: 5]
MAPLRPLLILALLAWVALADQESCKGRCTEGFNVDKKCQCDELCSYYQSC
CTDYTAECKPQVTRGDVFTMPEDEYTVYDDGEEKNNATVHEQVGGPSLTS
DLQAQSKGNPEQTPVLKPEEEAPAPEVGASKPEGIDSRPETLHPGRPQPP
AEEELCSGKPFDAFTDLKNGSLFAFRGQYCYELDEKAVRPGYPKLIRDVW
GIEGPIDAAFTRINCQGKTYLFKGSQYWRFEDGVLDPDYPRNISDGFDGI
PDNVDAALALPAHSYSGRERVYFFKGKQYWEYQFQHQPSQEECEGSSLSA
VFEHFAMMQRDSWEDIFELLFWGRTSAGTRQPQFISRDWHGVPGQVDAAM
AGRIYISGMAPRPSLAKKQRFRHRNRKGYRSQRGHSRGRNQNSRRPSRAT
WLSLFSSEESNLGANNYDDYRMDWLVPATCEPIQSVFFFSGDKYYRVNLR
TRRVDTVDPPYPRSIAQYWLGCPAPGHL

In certain embodiments, the VTN polypeptide comprises a signal peptide. In certain embodiments, the VTN polypeptide lacks a signal peptide. In certain embodiments, the VTN polypeptide comprises an amino acid sequence at 80% identical to the amino acid sequence set forth in SEQ ID NO: 6. In certain embodiments, the VTN polypeptide comprises an amino acid sequence at about 85%, at least about 90%, at least about 95%, at least about 96%, at least about 97%, at least about 98%, or at least about 99% identical to the amino acid sequence set forth in SEQ ID NO: 6. In certain embodiments, the VTN polypeptide comprises the amino acid sequence set forth in SEQ ID NO: 6. In certain embodiments, the VTN polypeptide consists of the amino acid sequence set forth in SEQ ID NO: 6. SEQ ID NO. 6 is provided below.

[SEQ ID NO: 6]
DQESCKGRCTEGFNVDKKCQCDELCSYYQSCCTDYTAECKPQVTRGDVFT
MPEDEYTVYDDGEEKNNATVHEQVGGPSLTSDLQAQSKGNPEQTPVLKPE
EEAPAPEVGASKPEGIDSRPETLHPGRPQPPAEEELCSGKPFDAFTDLKN
GSLFAFRGQYCYELDEKAVRPGYPKLIRDVWGIEGPIDAAFTRINCQGKT
YLFKGSQYWRFEDGVLDPDYPRNISDGFDGIPDNVDAALALPAHSYSGRE
RVYFFKGKQYWEYQFQHQPSQEECEGSSLSAVFEHFAMMQRDSWEDIFEL
LFWGRTSAGTRQPQFISRDWHGVPGQVDAAMAGRIYISGMAPRPSLAKKQ
RFRHRNRKGYRSQRGHSRGRNQNSRRPSRATWLSLESSEESNLGANNYDD
YRMDWLVPATCEPIQSVFFFSGDKYYRVNLRTRRVDTVDPPYPRSIAQYW
LGCPAPGHL

In certain embodiments, the VTN polypeptide is a VTN V65 subunit. In certain embodiments, the VTN polypeptide comprises an amino acid sequence at 80% identical to the amino acid sequence set forth in SEQ ID NO: 7. In certain embodiments, the VTN polypeptide comprises an amino acid sequence at about 85%, at least about 90%, at least about 95%, at least about 96%, at least about 97%, at least about 98%, or at least about 99% identical to the amino acid sequence set forth in SEQ ID NO: 7. In certain embodiments, the VTN polypeptide comprises the amino acid sequence set forth in SEQ ID NO: 7. In certain embodiments, the VTN polypeptide consists of the amino acid sequence set forth in SEQ ID NO: 7. SEQ ID NO. 7 is provided below.

[SEQ ID NO: 7]
DQESCKGRCTEGFNVDKKCQCDELCSYYQSCCTDYTAECKPQVTRGDVFT
MPEDEYTVYDDGEEKNNATVHEQVGGPSLTSDLQAQSKGNPEQTPVLKPE
EEAPAPEVGASKPEGIDSRPETLHPGRPQPPAEEELCSGKPFDAFTDLKN
GSLFAFRGQYCYELDEKAVRPGYPKLIRDVWGIEGPIDAAFTRINCQGKT
YLFKGSQYWRFEDGVLDPDYPRNISDGFDGIPDNVDAALALPAHSYSGRE
RVYFFKGKQYWEYQFQHQPSQEECEGSSLSAVFEHFAMMQRDSWEDIFEL
LFWGRTSAGTRQPQFISRDWHGVPGQVDAAMAGRIYISGMAPRPSLAKKQ
RFRHRNRKGYRSQRGHSRGRNQNSRRPSR

In certain embodiments, the VTN polypeptide is a VTN V10 subunit. In certain embodiments, the VTN polypeptide comprises an amino acid sequence at 80% identical to the amino acid sequence set forth in SEQ ID NO: 8. In certain embodiments, the VTN polypeptide comprises an amino acid sequence at about 85%, at least about 90%, at least about 95%, at least about 96%, at least about 97%, at least about 98%, or at least about 99% identical to the amino acid sequence set forth in SEQ ID NO: 8. In certain embodiments, the VTN polypeptide comprises the amino acid sequence set forth in SEQ ID NO: 8. In certain embodiments, the VTN polypeptide consists of the amino acid sequence set forth in SEQ ID NO: 8. SEQ ID NO. 8 is provided below.

[SEQ ID NO: 8]
ATWLSLESSEESNLGANNYDDYRMDWLVPATCEPIQSVFFFSGDKYYRVN
LRTRRVDTVDPPYPRSIAQYWLGCPAPGHL

In certain embodiments, the complement inhibitory protein is a complement component 1q subcomponent binding protein (C1QBP) polypeptide or a functional fragment thereof. In certain embodiments, the C1QBP polypeptide is a human C1QBP polypeptide. C1QBP is a multifunctional protein involved in immune response, energy homeostasis of cells as a plasma membrane receptor, and a nuclear, cytoplasmic, or mitochondrial protein.

In certain embodiments, the C1QBP polypeptide comprises an amino acid sequence at 80% identical to the amino acid sequence set forth in SEQ ID NO: 9. In certain embodiments, the C1QBP polypeptide comprises an amino acid sequence at about 85%, at least about 90%, at least about 95%, at least about 96%, at least about 97%, at least about 98%, or at least about 99% identical to the amino acid sequence set forth in SEQ ID NO: 9. In certain embodiments, the C1QBP polypeptide comprises the amino acid sequence set forth in SEQ ID NO: 9. In certain embodiments, the C1QBP polypeptide consists of the amino acid sequence set forth in SEQ ID NO: 9. SEQ ID NO. 9 is provided below.

[SEQ ID NO: 9]
MLPLLRCVPRVLGSSVAGLRAAAPASPFRQLLQPAPRLCTRPFGLLSVRA
GSERRPGLLRPRGPCACGCGCGSLHTDGDKAFVDFLSDEIKEERKIQKHK
TLPKMSGGWELELNGTEAKLVRKVAGEKITVTFNINNSIPPTFDGEEEPS
QGQKVEEQEPELTSTPNFVVEVIKNDDGKKALVLDCHYPEDEVGQEDEAE
SDIFSIREVSFQSTGESEWKDTNYTLNTDSLDWALYDHLMDFLADRGVDN
TFADELVELSTALEHQEYITFLEDLKSFVKSQ

In certain embodiments, the C1QBP polypeptide comprises a transit peptide. In certain embodiments, the C1QBP polypeptide lacks a transit peptide. In certain embodiments, the C1QBP polypeptide comprises an amino acid sequence at 80% identical to the amino acid sequence set forth in SEQ ID NO: 10. In certain embodiments, the C1QBP polypeptide comprises an amino acid sequence at about 85%, at least about 90%, at least about 95%, at least about 96%, at least about 97%, at least about 98%, or at least about 99% identical to the amino acid sequence set forth in SEQ ID NO: 10. In certain embodiments, the C1QBP polypeptide comprises the amino acid sequence set forth in SEQ ID NO: 10. In certain embodiments, the C1QBP polypeptide consists of the amino acid sequence set forth in SEQ ID NO: 10. SEQ ID NO. 10 is provided below.

[SEQ ID NO: 10]
LHTDGDKAFVDFLSDEIKEERKIQKHKTLPKMSGGWELELNGTEAKLVRK
VAGEKITVTFNINNSIPPTFDGEEEPSQGQKVEEQEPELTSTPNFVVEVI
KNDDGKKALVLDCHYPEDEVGQEDEAESDIFSIREVSFQSTGESEWKDTN
YTLNTDSLDWALYDHLMDFLADRGVDNTFADELVELSTALEHQEYITFLE
DLKSFVKSQ

In certain embodiments, the pharmaceutical composition comprises a CD59polypeptide and a SERPING1 polypeptide. In certain embodiments, the pharmaceutical composition comprises a CD59 polypeptide and a VTN polypeptide. In certain embodiments, the pharmaceutical composition comprises a CD59 polypeptide and a CIQBP polypeptide. In certain embodiments, the pharmaceutical composition comprises a SERPING1 polypeptide and a VTN polypeptide. In certain embodiments, the pharmaceutical composition comprises a SERPING1 polypeptide and a C1QBP polypeptide. In certain embodiments, the pharmaceutical composition comprises a VTN polypeptide and a C1QBP polypeptide.

In certain embodiments, the pharmaceutical composition comprises a CD59 polypeptide, a SERPING1 polypeptide, and a VTN polypeptide. In certain embodiments, the pharmaceutical composition comprises a CD59 polypeptide, a SERPING1 polypeptide, and a C1QBP polypeptide. In certain embodiments, the pharmaceutical composition comprises a SERPING1 polypeptide, a VTN polypeptide, and a C1QBP polypeptide. In certain embodiments, the pharmaceutical composition comprises a CD59 polypeptide, a SERPING1 polypeptide, a VTN polypeptide, and a C1QBP polypeptide.

In certain embodiments, the pharmaceutical composition further comprises at least one molecule capable of promoting neuronal generation. In certain embodiments, the molecule capable of promoting neuronal generation is a polypeptide. In certain embodiments, the polypeptide is selected from the group consisting of TIMP1, TIMP2, SEMA5A, FKBP4, VCL, LGALS1, CTSZ, PQBP1, TGFB1, LAMB1, PRPF19, LEPRE1, LRP1, VASP, YWHAH, SERPINF1, JAG1, GPC1, YWHAE, IQGAP1, ARHGDIA, SOD1, THY1, APP, DBN1, MAP1B, AXL, TWF2, COL3A1, ALCAM, YWHAG, DPYSL2, CALR, CSF1, FLRT2, CAPRIN1, NEO1, EPB41L3, DPYSL3, APOD, HMGB1, VAPA, CRKL, FN1, PDLIM7, SPTBN1, MAP4, ENAH, EZR, PTPRK, FLNA, HSP90AB1, SPTAN1, FAM129B, CTTN, ACTR2, EXT1, POSTN, XRCC5, SPOCK1, ITGB1, TRIOBP, LAMB2, RHOA, PRMT1, STMN1, SERPINE2, EIF4G1, DBNL, PTK7, WDR1, CFL1, MANF, DAG1, VIM, B2M, LDLR, VEGFA, YAP1, VEGFC, PDLIM5, CRABP2,and SPON2. In certain embodiments, the polypeptide is TIMP1. In certain embodiments, the polypeptide is TIMP2. In certain embodiments, the polypeptide is SEMA5A.

In certain embodiments, the pharmaceutical composition further comprises a TIMP1polypeptide or a functional fragment thereof. In certain embodiments, the TIMP1 polypeptide comprises an amino acid sequence at 80% identical to the amino acid sequence set forth in SEQ ID NO: 11. In certain embodiments, the TIMP1 polypeptide comprises an amino acid sequence at about 85%, at least about 90%, at least about 95%, at least about 96%, at least about 97%, at least about 98%, or at least about 99% identical to the amino acid sequence set forth in SEQ ID NO: 11. In certain embodiments, the TIMP1 polypeptide comprises the amino acid sequence set forth in SEQ ID NO: 11. In certain embodiments, the TIMP1 polypeptide consists of the amino acid sequence set forth in SEQ ID NO: 11. SEQ ID NO. 11 is provided below.

[SEQ ID NO: 11]
MAPFEPLASGILLLLWLIAPSRACTCVPPHPQTAFCNSDLVIRAKFVGTP
EVNQTTLYQRYEIKMTKMYKGFQALGDAADIRFVYTPAMESVCGYFHRSH
NRSEEFLIAGKLQDGLLHITTCSFVAPWNSLSLAQRRGFTKTYTVGCEEC
TVFPCLSIPCKLQSGTHCLWTDQLLQGSEKGFQSRHLACLPREPGLCTWQ
SLRSQIA

In certain embodiments, the pharmaceutical composition further comprises a TIMP2 polypeptide or a functional fragment thereof. In certain embodiments, the TIMP2 polypeptide comprises an amino acid sequence at 80% identical to the amino acid sequence set forth in SEQ ID NO: 12. In certain embodiments, the TIMP2 polypeptide comprises an amino acid sequence at about 85%, at least about 90%, at least about 95%, at least about 96%, at least about 97%, at least about 98%, or at least about 99% identical to the amino acid sequence set forth in SEQ ID NO: 12. In certain embodiments, the TIMP2 polypeptide comprises the amino acid sequence set forth in SEQ ID NO: 12. In certain embodiments, the TIMP2 polypeptide consists of the amino acid sequence set forth in SEQ ID NO: 12. SEQ ID NO. 12 is provided below.

[SEQ ID NO: 12]
MGAAARTLRLALGLLLLATLLRPADACSCSPVHPQQAFCNADVVIRAKAV
SEKEVDSGNDIYGNPIKRIQYEIKQIKMFKGPEKDIEFIYTAPSSAVCGV
SLDVGGKKEYLIAGKAEGDGKMHITLCDFIVPWDTLSTTQKKSLNHRYQM
GCECKITRCPMIPCYISSPDECLWMDWVTEKNINGHQAKFFACIKRSDGS
CAWYRGAAPPKQEFLDIEDP

In certain embodiments, the pharmaceutical composition further comprises a SEMA5A polypeptide or a functional fragment thereof. In certain embodiments, the SEMA5A polypeptide comprises an amino acid sequence at 80% identical to the amino acid sequence set forth in SEQ ID NO: 13. In certain embodiments, the SEMA5A polypeptide comprises an amino acid sequence at about 85%, at least about 90%, at least about 95%, at least about 96%, at least about 97%, at least about 98%, or at least about 99% identical to the amino acid sequence set forth in SEQ ID NO: 13. In certain embodiments, the SEMA5A polypeptide comprises the amino acid sequence set forth in SEQ ID NO: 13. In certain embodiments, the SEMA5A polypeptide consists of the amino acid sequence set forth in SEQ ID NO: 13. SEQ ID NO. 13 is provided below.

[SEQ ID NO: 13]
MKGTCVIAWLFSSLGLWRLAHPEAQGTTQCQRTEHPVISYKEIGPWLREF
RAKNAVDFSQLTFDPGQKELVVGARNYLFRLQLEDLSLIQAVEWECDEAT
KKACYSKGKSKEECQNYIRVLLVGGDRLFTCGTNAFTPVCTNRSLSNLTE
IHDQISGMARCPYSPQHNSTALLTAGGELYAATAMDFPGRDPAIYRSLGI
LPPLRTAQYNSKWLNEPNFVSSYDIGNFTYFFFRENAVEHDCGKTVFSRA
ARVCKNDIGGRFLLEDTWTTFMKARLNCSRPGEVPFYYNELQSTFFLPEL
DLIYGIFTTNVNSIAASAVCVFNLSAIAQAFSGPFKYQENSRSAWLPYPN
PNPHFQCGTVDQGLYVNLTERNLQDAQKFILMHEVVQPVTTVPSFMEDNS
RFSHVAVDVVQGREALVHIIYLATDYGTIKKVRVPLNQTSSSCLLEEIEL
FPERRREPIRSLQILHSQSVLFVGLREHVVKIPLKRCQFYRTRSTCIGAQ
DPYCGWDVVMKKCTSLEESLSMTQWEQSISACPTRNLTVDGHFGVWSPWT
PCTHTDGSAVGSCLCRTRSCDSPAPQCGGWQCEGPGMEIANCSRNGGWTP
WTSWSPCSTTCGIGFQVRQRSCSNPTPRHGGRVCVGQNREERYCNEHLLC
PPHMFWTGWGPWERCTAQCGGGIQARRRICENGPDCAGCNVEYQSCNTNP
CPELKKTTPWTPWTPVNISDNGGHYEQRFRYTCKARLADPNLLEVGRQRI
EMRYCSSDGTSGCSTDGLSGDFLRAGRYSAHTVNGAWSAWTSWSQCSRDC
SRGIRNRKRVCNNPEPKYGGMPCLGPSLEYQECNILPCPVDGVWSCWSPW
TKCSATCGGGHYMRTRSCSNPAPAYGGDICLGLHTEEALCNTQPCPESWS
EWSDWSECEASGVQVRARQCILLFPMGSQCSGNTTESRPCVFDSNFIPEV
SVARSSSVEEKRCGEFNMFHMIAVGLSSSILGCLLTLLVYTYCQRYQQQS
HDATVIHPVSPAPLNTSITNHINKLDKYDSVEAIKAFNKNNLILEERNKY
FNPHLTGKTYSNAYFTDLNNYDEY

Additionally or alternatively, the pharmaceutical composition further comprises at least one molecule capable of promoting neuronal projection development and/or axon guidance. In certain embodiments, the molecule capable of promoting neuronal projection development and/or axon guidance is a polypeptide. In certain embodiments, the polypeptide is selected from the group consisting of NEO1, APP, FKBP4, VCL, LGALS1, CTSZ, PQBP1, TGFB1, LAMB1, PRPF19, LEPRE1, LRP1, VASP, YWHAH, SERPINF1, JAG1, TIMP2, GPC1, YWHAE, IQGAP1, ARHGDIA, SOD1, THY1, APP, DBN1, MAP1B, AXL, TWF2, COL3A1, ALCAM, YWHAG, DPYSL2, CALR, CSF1, FLRT2, CAPRIN1, EPB41L3, DPYSL3, APOD, HMGB1, VAPA, CRKL, FN1, PDLIM7, SPTBN1, MAP4, ENAH, EZR, PTPRK, FLNA, HSP90AB1, SPTAN1, FAM129B, CTTN, ACTR2, EXT1, POSTN, SEMASA, XRCC5, SPOCK1, ITGB1, TRIOBP, LAMB2, RHOA, PRMT1, STMN1, SERPINE2, EIF4G1, DBNL, PTK7, WDR1, CFL1, MANF, DAG1, VIM, B2M, LDLR, VEGFA, YAP1, VEGFC, PDLIM5, CRABP2, SPON2. In certain embodiments, the polypeptide is NEO1. In certain embodiments, the polypeptide is APP.

In certain embodiments, the pharmaceutical composition further comprises a NEO1 polypeptide or a functional fragment thereof. In certain embodiments, the NEO1 polypeptide comprises an amino acid sequence at 80% identical to the amino acid sequence set forth in SEQ ID NO: 14. In certain embodiments, the NEO1 polypeptide comprises an amino acid sequence at about 85%, at least about 90%, at least about 95%, at least about 96%, at least about 97%, at least about 98%, or at least about 99% identical to the amino acid sequence set forth in SEQ ID NO: 14. In certain embodiments, the NEO1 polypeptide comprises the amino acid sequence set forth in SEQ ID NO: 14. In certain embodiments, the NEO1 polypeptide consists of the amino acid sequence set forth in SEQ ID NO: 14. SEQ ID NO. 14 is provided below.

[SEQ ID NO: 14]
MAAERGARRLLSTPSFWLYCLLLLGRRAPGAAAARSGSAPQSPGASIRTF
TPFYFLVEPVDTLSVRGSSVILNCSAYSEPSPKIEWKKDGTFLNLVSDDR
RQLLPDGSLFISNVVHSKHNKPDEGYYQCVATVESLGTIISRTAKLIVAG
LPRFTSQPEPSSVYAGNNAILNCEVNADLVPFVRWEQNRQPLLLDDRVIK
LPSGMLVISNATEGDGGLYRCVVESGGPPKYSDEVELKVLPDPEVISDLV
FLKQPSPLVRVIGQDVVLPCVASGLPTPTIKWMKNEEALDTESSERLVLL
AGGSLEISDVTEDDAGTYFCIADNGNETIEAQAELTVQAQPEFLKQPTNI
YAHESMDIVFECEVTGKPTPTVKWVKNGDMVIPSDYFKIVKEHNLQVLGL
VKSDEGFYQCIAENDVGNAQAGAQLIILEHAPATTGPLPSAPRDVVASLV
STRFIKLTWRTPASDPHGDNLTYSVFYTKEGIARERVENTSHPGEMQVTI
QNLMPATVYIFRVMAQNKHGSGESSAPLRVETQPEVQLPGPAPNLRAYAA
SPTSITVTWETPVSGNGEIQNYKLYYMEKGTDKEQDVDVSSHSYTINGLK
KYTEYSFRVVAYNKHGPGVSTPDVAVRTLSDVPSAAPQNLSLEVRNSKSI
MIHWQPPAPATQNGQITGYKIRYRKASRKSDVTETLVSGTQLSQLIEGLD
RGTEYNFRVAALTINGTGPATDWLSAETFESDLDETRVPEVPSSLHVRPL
VTSIVVSWIPPENQNIVVRGYAIGYGIGSPHAQTIKVDYKQRYYTIENLD
PSSHYVITLKAFNNVGEGIPLYESAVTRPHTDTSEVDLFVINAPYTPVPD
PTPMMPPVGVQASILSHDTIRITWADNSLPKHQKITDSRYYTVRWKTN
IPANTKYKNANATTLSYLVTGLKPNTLYEFSVMVTKGRRSSTWSMTAHGT
TFELVPTSPPKDVTVVSKEGKPKTIIVNWQPPSEANGKITGYIIYYSTDV
NAEIHDWVIEPVVGNRLTHQIQELTLDTPYYFKIQARNSKGMGPMSEAVQ
FRTPKADSSDKMPNDQASGSGGKGSRLPDLGSDYKPPMSGSNSPHGSPTS
PLDSNMLLVIIVSVGVITIVVVVIIAVFCTRRTTSHQKKKRAACKSVNGS
HKYKGNSKDVKPPDLWIHHERLELKPIDKSPDPNPIMTDTPIPRNSQDIT
PVDNSMDSNIHQRRNSYRGHESEDSMSTLAGRRGMRPKMMMPFDSQPPQP
VISAHPIHSLDNPHHHFHSSSLASPARSHLYHPGSPWPIGTSMSLSDRAN
STESVRNTPSTDTMPASSSQTCCTDHQDPEGATSSSYLASSQEEDSGQSL
PTAHVRPSHPLKSFAVPAIPPPGPPTYDPALPSTPLLSQQALNHHIHSVK
TASIGTLGRSRPPMPVVVPSAPEVQETTRMLEDSESSYEPDELTKEMAHL
EGLMKDLNAITTA

In certain embodiments, the pharmaceutical composition further comprises an APP polypeptide or a functional fragment thereof. In certain embodiments, the APP polypeptide comprises an amino acid sequence at 80% identical to the amino acid sequence set forth in SEQ ID NO: 15. In certain embodiments, the APP polypeptide comprises an amino acid

Active 104100082.1.DOCX 21 sequence at about 85%, at least about 90%, at least about 95%, at least about 96%, at least about 97%, at least about 98%, or at least about 99% identical to the amino acid sequence set forth in SEQ ID NO: 15. In certain embodiments, the APP polypeptide comprises the amino acid sequence set forth in SEQ ID NO: 152. In certain embodiments, the APP polypeptide consists of the amino acid sequence set forth in SEQ ID NO: 15. SEQ ID NO. 15 is provided below.

[SEQ ID NO: 15]
MLPGLALLLLAAWTARALEVPTDGNAGLLAEPQIAMFCGRLNMHMNVQNG
KWDSDPSGTKTCIDTKEGILQYCQEVYPELQITNVVEANQPVTIQNWCKR
GRKQCKTHPHFVIPYRCLVGEFVSDALLVPDKCKFLHQERMDVCETHLHW
HTVAKETCSEKSTNLHDYGMLLPCGIDKFRGVEFVCCPLAEESDNVDSAD
AEEDDSDVWWGGADTDYADGSEDKVVEVAEEEEVAEVEEEEADDDEDDED
GDEVEEEAEEPYEEATERTTSIATTTTTTTESVEEVVREVCSEQAETGPC
RAMISRWYFDVTEGKCAPFFYGGCGGNRNNFDTEEYCMAVCGSAMSQSLL
KTTQEPLARDPVKLPTTAASTPDAVDKYLETPGDENEHAHFQKAKERLEA
KHRERMSQVMREWEEAERQAKNLPKADKKAVIQHFQEKVESLEQEAANER
QQLVETHMARVEAMLNDRRRLALENYITALQAVPPRPRHVFNMLKKYVRA
EQKDRQHTLKHFEHVRMVDPKKAAQIRSQVMTHLRVIYERMNQSLSLLYN
VPAVAEEIQDEVDELLQKEQNYSDDVLANMISEPRISYGNDALMPSLTET
KTTVELLPVNGEFSLDDLQPWHSFGADSVPANTENEVEPVDARPAADRGL
TTRPGSGLTNIKTEEISEVKMDAEFRHDSGYEVHHQKLVFFAEDVGSNKG
AIIGLMVGGVVIATVIVITLVMLKKKQYTSIHHGVVEVDAAVTPEERHLS
KMQQNGYENPTYKFFEQMQN

In certain non-limited embodiments, the pharmaceutical composition can include secretome or conditioned media harvested from a human corneal stromal stem cell (CSSC). For example, but without any limitation, CSSCs can be cultured as clonal culture and be used for harvesting secretome as illustrated in Example 1 below. As used herein, the term “secretome” refers to an array of cytokines, growth factors, small RNAs, non-coding RNAs, ECM mediators, and proteins secreted by a cell. For example, but without any limitation, a human corneal stromal stem cell (CSSC) secretome can include a set of proteins expressed by the CSSCs and secreted into the extracellular space.

In certain embodiments, CSSCs can be cultured with at least about 1 ml, at least about 3 ml, at least about 5 ml, at least about 10 ml, at least about 25 ml, at least about 50 ml, at least about 75 ml, or at least about 100 ml of a basal media for harvesting secretome. In certain embodiments, the volume of the basal media can be from about 1 ml to about 10 ml, from about 1 ml to about 25 ml, from about 1 ml to about 50 ml, from about 1 ml to about 100 ml, from about 10 ml to about 50 ml, from about 10 ml to about 100 ml, or from about 50 ml to about 100 ml. In certain embodiments, the number of CSSCs cultured for harvesting secretome can be at least about 1×10⁵, at least about 1×10⁶, at least about 1×10⁷, at least about 1×10⁸, at least about 1×10⁹, or at least about 1×10¹⁰. In certain embodiments, the number of CSSCs cultured for harvesting secretome can be from about 1×10⁵ to about 1×10⁶, from about 1×10⁶ to about 1×10⁷, from about 1×10⁷ to about 1×10⁸, from about 1×10⁸ to about 1×10⁹, or from about 1×10⁹ to about 1×10¹⁰. In certain embodiments, CSSCs can be cultured to the log phase and incubated with basal media without serum and growth factors at about 60% to about 70% confluence for a predetermined period. In certain embodiments, the predetermined period can be at least about 1 hour, at least about 3 hours, at least about 5 hours, at least about 10 hours, at least about 12 hours, at least about 24 hours, at least about 48 hours, or at least about 72 hours.

In certain embodiments, the harvested secretome can be further concentrated. In certain embodiments, the harvested secretome can be concentrated up to about 2 times (2×), about five times (5×), about ten times (10×), about twenty times (20×), about twenty-five times (25×), about fifty times (50×), about seventy-five times (75×), or about one hundred times (100×) of the initial volume. For example, but without any limitation, the harvested secretome can be concentrated using a centrifugal filter up to twenty-five times (25×) of the initial volume. In certain embodiments, the concentrated secretome can be stored at about −80° C.

In certain embodiments, the disclosed secretome can be filtered to remove any cell debris in order to reduce cytotoxicity, immune responses, and/or side effects.

In certain embodiments, the CSSC secretome can include a set of proteins that relate to axon guidance, neurogenesis, neuron death, neuron apoptosis, or combinations thereof. For example, but without any limitation, the CSSC secretome can include complement inhibitors proteins (e.g., CD59, SERPING1, VTN, and C1QBP), proteins related to the axon guidance pathways (e.g., SEMA5A), proteins involved in neurogenesis (e.g., NEO1), and neuron projection maintenance and development (e.g., APP).

In certain non-limiting embodiments, the pharmaceutical compositions disclosed herein include a pharmaceutically acceptable carrier. “Pharmaceutically acceptable,” as used herein, includes any carrier which does not interfere with the effectiveness of the biological activity of the active ingredients, e.g., a polypeptide, and that is not toxic to the patient to whom it is administered. Non-limiting examples of suitable pharmaceutical carriers include phosphate-buffered saline solutions, water, emulsions, such as oil/water emulsions, various types of wetting agents, and sterile solutions. Additional non-limiting examples of pharmaceutically acceptable carriers can include gels, bioabsorbable matrix materials, implantation clements containing the inhibitor, and/or any other suitable vehicle, delivery, or dispensing means or material. Such carriers can be formulated by conventional methods and can be administered to the subject.

In certain embodiments, the pharmaceutical compositions of the present disclosure can be formulated using pharmaceutically acceptable carriers well known in the art that are suitable for parenteral administration, e.g., intraocular administration, topical administration intravenous administration, intraarterial administration, intrathecal administration, intranasal administration, intramuscular administration, subcutaneous administration, and intracisternal administration. In certain embodiments, the pharmaceutical composition is formulated for ocular administration (e.g., ocular infusion). For example, but not by way of limitation, the pharmaceutical formulation can be formulated as solutions, suspensions, or emulsions.

In certain non-limiting embodiments, the pharmaceutical compositions of the present disclosure can be formulated using pharmaceutically acceptable carriers and excipients well known in the art that are suitable for ocular administration. Non-limiting examples of excipients include inert fillers or diluents, such as sucrose, sorbitol, sugar, mannitol, microcrystalline cellulose, starches, including potato starch, calcium carbonate, sodium chloride, lactose, calcium phosphate, calcium sulfate or sodium phosphate; crumbling agents and disintegrants, for example, cellulose derivatives, including microcrystalline cellulose, starches, including potato starch, alginates or alginic acid and chitosan; binding agents, for example, sucrose, glucose, sorbitol, acacia, alginic acid, sodium alginate, gelatin, starch, microcrystalline cellulose, aluminum magnesium silicate, sodium carboxymethylcellulose, methylcellulose, hydroxypropyl methylcellulose, ethyl cellulose, polyvinylpyrrolidone, polyvinyl acetate or polyethylene glycol, and chitosan; lubricating agents, including glidants and antiadhesive agents, for example, magnesium stearate, zinc stearate, stearic acid, silicas, hydrogenated vegetable oils or talc.

In certain embodiments, the pharmaceutical compositions are formulated as an ophthalmic formulation for administration to the eye. In certain embodiments, the pharmaceutical compositions include ophthalmic buffer solutions such as boric acid vehicles and Sorensen's modified phosphate buffer. In certain embodiments, the boric acid vehicle is a 1.9% solution of boric acid in purified water or sterile water. In certain embodiments, the boric acid vehicle is isotonic with tears. In certain embodiments, the boric acid vehicle has a pH of about 5.

In certain embodiments, the pharmaceutical compositions include preservatives. Such preservatives are used in order to prevent the growth of or to destroy microorganisms accidentally introduced when the container is opened during use. Preservatives encompassed by the present disclosure include, for example and without any limitation, benzyl alcohol and parabens.

In certain embodiments, the pharmaceutical compositions include an antioxidant. Non-limiting examples of antioxidants include sodium metabisulfite and sodium bisulfite.

In certain embodiments, the pharmaceutical composition can be formulated to release the active ingredients (e.g., any of the polypeptides disclosed herein) immediately upon administration. Alternatively, the pharmaceutical compositions can be formulated to release the active ingredients (e.g., any of the polypeptides disclosed herein) at any predetermined time or time period after administration. Such types of compositions are generally known as controlled release formulations, which include (i) formulations that create substantially constant concentrations of the active ingredients (e.g., any of the polypeptides disclosed herein) within the subject over an extended period of time; (ii) formulations that after a predetermined lag time create substantially constant concentrations of the active ingredients (e.g., any of the polypeptides disclosed herein) within the subject over an extended period of time; (iii) formulations that sustain the action of the active ingredients (e.g., any of the polypeptides disclosed herein) during a predetermined time period by maintaining a relatively constant, effective level of the active ingredients (e.g., any of the polypeptides disclosed herein) in the body with concomitant minimization of undesirable side effects; (iv) formulations that localize action of active ingredients (e.g., any of the polypeptides disclosed herein), e.g., spatial placement of a controlled release composition adjacent to or in the disease, e.g., corneal cells; (v) formulations that achieve convenience of dosing, e.g., administering the composition once per week or once every two weeks; and (vi) formulations that target the action of the active ingredients (e.g., any of the polypeptides disclosed herein) by using carriers or chemical derivatives to deliver the platelet inhibitor to a particular target cell type or a particular target tissue type. In certain embodiments, controlled release is obtained by an appropriate selection of various formulation parameters and ingredients, including, e.g., various types of controlled release compositions and coatings.

In certain embodiments, the pharmaceutical compositions suitable for use in the present disclosure can include compositions, where the active ingredients (e.g., any of the polypeptides disclosed herein) disclosed herein, are contained in a therapeutically effective amount. A “therapeutically effective amount” refers to an amount that is able to prevent and/or treat a corneal wound and reduce corneal scarring. The therapeutically effective amount of an active ingredient can vary depending on the active ingredient, e.g., a polypeptide disclosed herein, the formulation used, the way of administration, and the age, weight, etc., of the subject to be treated. In certain embodiments, a patient can receive a therapeutically effective amount of a pharmaceutical composition disclosed herein as a single dose or multiple administrations of two or more doses, which can depend on the dosage and frequency as required and tolerated by the patient. In certain embodiments, the provided methods involve administering the compositions at effective amounts, e.g., therapeutically effective amounts.

In certain embodiments, the presently disclosed pharmaceutical compositions can be formulated as a hydrogel. As used herein, the term “hydrogel” refers to a three-dimensional, predominantly hydrophilic polymeric network comprising a large quantity of water, formed by chemical or physical crosslinking of natural or synthetic homopolymers, copolymers, or oligomers. Hydrogels can be formed through crosslinking polyethylene glycols (considered to be synonymous with polyethylene oxides), polypropylene glycols, poly(N-vinylpyrrolidone), polymethacrylates, polyphosphazenes, polylactides, polyacrylamides, polyglycolates, polyethylene imines, agarose, dextran, gelatin, collagen, polylysine, chitosans, alginates, hyaluronans, pectin, carrageenan. In certain embodiments, the hydrogel is a mesoporous hydrogel. As used herein, the term “mesoporous hydrogel” refers to a hydrogel having pores between about 1 nm and about 100 nm in diameter. These pores in the mesoporous hydrogels are sufficiently large to allow for free diffusion of biological molecules such as the polypeptides of the pharmaceutical compositions disclosed herein. In certain embodiments, the hydrogel is a macroporous hydrogel. As used herein, the term “macroporous hydrogel” refers to a hydrogel having pores greater than about 100 nm in diameter. In certain embodiments, the hydrogel is a microporous hydrogel. As used herein, the term “microporous hydrogel” refers to a hydrogel having pores less than about 1 nm in diameter.

In certain embodiments, the hydrogel is formed using reactive polymers and reactive oligomers. In certain embodiments, the reactive polymers and reactive oligomers include functional groups that are reactive toward other functional groups. In certain embodiments, the reactive polymers and reactive oligomers react under mild conditions in order to preserve the stability of the polypeptides of the pharmaceutical composition. Non-limiting examples of functional groups of the reactive polymers and reactive oligomers include malcimides, thiols or protected thiols, alcohols, acrylates, acrylamides, amines or protected amines, carboxylic acids or protected carboxylic acids, azides, alkynes including cycloalkynes, 1,3-dienes including cyclopentadienes and furans, alpha-halocarbonyls, and N-hydroxysuccinimidyl, N-hydroxysulfosuccinimidyl, or nitrophenyl esters or carbonates.

In certain embodiments, the hydrogel is biodegradable. By being biodegradable, a loses its structural integrity through the cleavage of component chemical bonds under physiological conditions of pH and temperature. In certain embodiments, the hydrogel is enzymatically catalyzed. In certain embodiments, the hydrogel includes thrombin, fibrinogen, or a combination thereof.

### 3. Contact Lenses

In certain embodiments, the present disclosure also provides contact lenses comprising the pharmaceutical compositions disclosed herein. In certain embodiments, the contact lenses can be hard lenses, semi-rigid lenses, hybrid lenses, smart lenses, or soft lenses. In certain embodiments, the contact lenses are soft contact lenses. In certain embodiments, the contact lenses are hydrogel lenses.

In certain embodiments, the hydrogel lenses are conventional hydrogel lenses. As used herein, the term “conventional hydrogel lenses” refers to contact lenses including polymeric networks made from components without any siloxy, siloxane, or carbosiloxane groups. In certain embodiments, conventional hydrogel lenses include a hydrogel prepared from reactive mixtures comprising hydrophilic monomers that, when polymerized, form the base material of the lens. Non-limiting examples of hydrophilic monomers 2-hydroxyethyl methacrylate (“HEMA”), N,N-dimethylacrylamide (DMA), and N-vinyl pyrrolidone (“NVP”). Conventional hydrogel lenses can also include a hydrogel be formed from polyvinyl alcohol.

In certain embodiments, the conventional hydrogel lenses include, but without any limitation, ctafilcon, genfilcon, hilafilcon, lenefilcon, nelfilcon, nesofilcon, ocufilcon, omafilcon, polymacon, and vifilcon, and variants thereof. In certain embodiments, conventional hydrogel lenses include a coating. In certain embodiments, the coating is the same or different material from a substrate. In certain embodiments, conventional hydrogel lenses include additives (e.g., polyvinyl pyrrolidone). In certain embodiments, conventional hydrogel lenses include additives comonomers (e.g., phosphoryl choline or methacrylic acid).

In certain embodiments, the hydrogel lenses are silicone hydrogel lenses. As used herein, the term “silicone hydrogel lenses” refers to contact lenses including a polymeric network made from at least one hydrophilic component and at least one silicone-containing component that, when polymerized, form the base material of the lens. Non-limiting examples of suitable families of hydrophilic components present in the reactive mixture include (meth) acrylates, styrenes, vinyl ethers, (meth) acrylamides, N-vinyl lactams, N-vinyl amides, N-vinyl imides, N-vinyl ureas, O-vinyl carbamates, O-vinyl carbonates, other hydrophilic vinyl compounds, and mixtures thereof. Non-limiting examples of suitable silicone-containing components include at least one polymerizable group (e.g., a (meth)acrylate, a styryl, a vinyl ether, a (meth)acrylamide, an N-vinyl lactam, an N-vinylamide, an O-vinylcarbamate, an O-vinylcarbonate, a vinyl group, or mixtures of the foregoing), at least one siloxane group, and one or more linking groups connecting the polymerizable group(s) to the siloxane group(s). In certain embodiments, the silicone-containing components include from about 1 to about 220 siloxane repeat units. In certain embodiments, the silicone-containing components include at least one fluorine atom.

Examples of silicone hydrogel base lens materials include, but without any limitation, acquafilcon, asmofilcon, balafilcon, comfilcon, delefilcon, enfilcon, fanfilcon, formofilcon, galyfilcon, lotrafilcon, narafilcon, riofilcon, samfilcon, senofilcon (including senofilcon A or senofilcon C), somofilcon, stenfilcon, and variants thereof. In certain embodiments, the silicone hydrogel lenses include a coating. In certain embodiments, the coating is the same or different material from the substrate.

In certain embodiments, the contact lenses can be daily-wear disposable contact lenses. In certain embodiments, the contact lenses can be daily-wear reusable contact lenses (e.g., monthly lenses). In certain embodiments, the contact lenses encompassed by the present disclosure also provide vision correction (e.g., prescription lenses).

### 4. Methods of Treatment

The present disclosure relates to methods for preventing, reducing the risk of, and/or treating corneal scarring. The present disclosure provides methods for preventing, reducing the risk of, and/or treating corneal scarring in a subject by inhibiting the complement system. As described in detail in the Example section below, the studies presented in the present disclosure indicate that the inhibition of the complement system, along with the promotion of neuronal generation, neuronal projection development, and/or axon guidance, can be used to prevent and/or treat corneal scarring.

The cornea is the most anterior part of the eye and provides the most refractive power of the eye. Corneal transparency is important for vision (Kumar et al., Prog Retin Eye Res 10.1016/j.preteyeres.2021.101011, 101011 (2021)). The corneal epithelium, the outermost layer of the cornea, is typically able to heal on its own after damage provided the limbal stem cells are not depleted. When a corneal wound reaches the Bowman's membrane, it ultimately causes damage to the corneal stroma, a mesenchymal tissue making up the thickest layer of the cornea (Park et al., Proc Natl Acad Sci U S A 10.1073/pnas.1912260116 (2019)). Corneal stromal injuries usually cause permanent haze and scarring, therefore leading to vision loss.

During corneal wounds, many growth factors and cytokines are activated for wound healing which contribute to keratocytes transformation into fibroblasts and myofibroblasts and excess deposition of extracellular matrix (ECM) proteins and fibrosis (Ljubimov et al., Prog Retin Eye Res 49, 17-45 (2015)). The cytokines also induce inflammatory and immune cell infiltration into the cornea (Ju et al., Front Cardiovasc Med 8, 649124 (2021)).). The inflammation also causes corneal cell death and sensory neuron death which compromise corneal transparency and result in vision impairment, even blindness (Yun et al., Immunity 53, 10501062 e1055 (2020)).

As used herein, the term “corneal scarring” refers to any opacity or irregularity on or within the corneal surface that can compromise its ability to transmit and reflect light correctly. In certain embodiments, corneal scarring impairs vision. In certain embodiments, corneal scarring in the central cornea impairs vision.

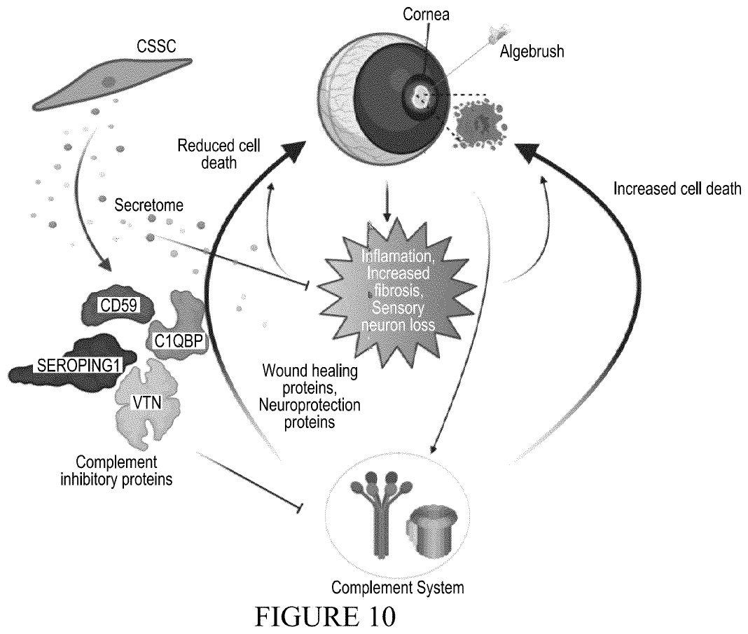

FIG. 10 illustrates the pathways regulated by the methods disclosed herein. Induction of a corneal wound increases inflammation and fibrosis which result in increased cell death in the wounded corneas. A corneal wound also increases the death of sensory neurons in the cornea which enhances corneal opacity. Neuroprotective proteins present in the presently disclosed pharmaceutical compositions related to neurogenesis, neuron projection development, and neuron differentiation rescue the neuronal loss in the wounded corneas. Also, proteins related to wound healing in the presently disclosed pharmaceutical compositions provide support for scarless wound healing. Complement inhibitory proteins present in the presently disclosed pharmaceutical compositions like CD59, SERPING1, C1QBP, and vitronectin (VTN) inhibit the complement system, rescuing the corneal cell death, and promoting scarless corneal wound healing.

In certain non-limiting embodiments, the present disclosure provides for a method of preventing and/or treating corneal scarring in a subject. In certain embodiments, the method can include administering a therapeutically effective amount of a pharmaceutical composition disclosed herein to the subject. In certain embodiments, the method can include administering a contact lens comprising a pharmaceutical composition disclosed herein to the subject. In certain embodiments, administration of the pharmaceutical composition improves the wound healing of corneal scarring. In certain embodiments, the subject was known to have corneal scarring prior to treatment. In certain non-limiting embodiments, the subject was not known to have corneal scarring prior to treatment.

In certain embodiments, the present disclosure provides methods for reducing the risk of a subject that had corneal scarring from developing new corneal scarring, which can include administering a therapeutically effective amount of a pharmaceutical composition to the subject. In certain embodiments, methods for reducing the risk of a subject that had corneal scarring from developing new corneal scarring include administering contact lenses comprising a pharmaceutical composition to the subject.

Methods disclosed herein can be used for treating any corneal scarring. In certain embodiments, methods disclosed herein can be used for treating a nebula. As used herein, the term “nebula” refers to a faint opacity of the cornea that remains after an ulcer has healed. In certain embodiments, methods disclosed herein can be used for treating maculopathy. As used herein, the term “maculopathy” refers to any abnormality of the macula of the eye. For example, bull's-eye maculopathy describes the appearance of the macula in some toxic conditions (e.g. chloroquine toxicity) and some hereditary disorders of the macula. In certain embodiments, methods disclosed herein can be used for treating a leucoma. As used herein, the term “leucoma” refers to a white opacity occurring in the cornea. Most leucomas result from scarring after corneal inflammation or ulceration. In certain embodiments, leucomas can be congenital and/or associated with other abnormalities of the eye.

In certain embodiments, a pharmaceutical composition can be administered to a subject at a dose of about 0.05 μg/day to about 100 μg/day. In certain embodiments, a subject can be administered up to about 2,000 μg of the pharmaceutical composition in a single dose or as a total daily dose. For example, but not by way of limitation, a subject can be administered up to about 1,950 μg, up to about 1,900 μg, up to about 1,850 μg, up to about 1,800 μg, up to about 1,750 μg, up to about 1,700 μg, up to about 1,650 μg, up to about 1,600 μg, up to about 1,550 μg, up to about 1,500 μg, up to about 1,450 μg, up to about 1,400 μg, up to about 1,350 μg, up to about 1,300 μg, up to about 1,250 μg, up to about 1,200 μg, up to about 1,150 μg, up to about 1,100 μg, up to about 1,050 μg, up to about 1,000 μg, up to about 950 μg, up to about 900 μg, up to about 850 μg, up to about 800 μg, up to about 750 μg, up to about 700 μg, up to about 650 μg, up to about 600 μg, up to about 550 μg, up to about 500 μg, up to about 450 μg, up to about 400 μg, up to about 350 μg, up to about 300 μg, up to about 250 μg, up to about 200 μg, up to about 150 μg, up to about 100 μg, up to about 50 μg or up to about 25 μg of the pharmaceutical composition in a single dose or as a total daily dose. In certain embodiments, the subject can be administered from about 50 to about 1,000 μg of the pharmaceutical composition in a single dose or a total daily dose. In certain embodiments, a subject can be administered about 1,000 μg of the pharmaceutical composition in a single dose or as a total daily dose. In certain embodiments, a subject can be administered about 25 μg or more of the pharmaceutical composition in a single dose or as a total daily dose. In certain embodiments, a subject can be administered about 1,000 μg of the pharmaceutical composition in a single dose or as a total daily dose. In certain embodiments, a subject can be administered about 25 μg or more of the pharmaceutical composition in a single dose or as a total daily dose.

It is to be understood that, for any particular subject, specific dosage regimes should be adjusted over time according to the individual need and the professional judgment of the person administering or supervising the administration of the pharmaceutical composition. For example, the dosage of the pharmaceutical composition can be increased if the lower dose does not provide sufficient activity in the treatment of a disease or condition described herein (e.g., corneal scarring). Alternatively, the dosage of the composition can be decreased if the disease (e.g., corneal scarring) is reduced, no longer detectable, or eliminated.

In certain embodiments, the pharmaceutical composition can be administered once a day, twice a day, once a week, twice a week, three times a week, four times a week, five times a week, six times a week, once every two weeks, once a month, twice a month, once every other month or once every third month. In certain embodiments, the pharmaceutical composition can be administered twice a week. In certain embodiments, the pharmaceutical composition can be administered once a week. In certain embodiments, the pharmaceutical composition can be administered two times a week for about four weeks and then administered once a week for the remaining duration of the treatment. In certain embodiments, a subject can be administered up to about 1,000 μg of the pharmaceutical composition in a single dose or as a total daily dose two times a week.

In certain embodiments, the period of treatment can be at least one day, at least one weck, at least one month, at least two months, at least three months, at least four months, at least five months, or at least six months. In certain embodiments, the pharmaceutical composition can be administered until the corneal scarring is no longer detectable.

### 5. Kits

The present disclosure provides kits for use in the disclosed methods. In certain embodiments, a kit can include a container that the pharmaceutical composition disclosed herein. In certain embodiments, the container can include a single dose of the pharmaceutical composition or multiple doses of the pharmaceutical composition. A container can be any receptacle and closure suitable for storing, shipping, dispensing, and/or handling a pharmaceutical product.

In certain embodiments, the kit can further include a second container that includes a solvent, carrier, and/or solution for diluting and/or resuspending the pharmaceutical composition. For example, but not by way of limitation, the second container can include sterile water.

In certain embodiments, the kits include a sterile container that contains the pharmaceutical composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

In certain embodiments, the kit can further include instructions for administering the pharmaceutical composition. The instructions can include information about the use of the pharmaceutical composition for treating corneal scarring. In certain embodiments, the instructions include at least one of the following: description of the pharmaceutical composition; dosage schedule and administration for treating the corneal scarring; precautions; warnings; indications; counter-indications; over dosage information; adverse reactions; animal pharmacology; clinical studies; and/or references. The instructions can be printed directly on the container (when present), as a label applied to the container, or as a separate sheet, pamphlet, card, or folder supplied in or with the container. For example, but not by way of limitation, the instructions can describe the method for administration and the dosage amount. In certain embodiments, the instructions indicate that the pharmaceutical composition thereof can be used for ocular administration. In certain embodiments, the instructions can indicate that the pharmaceutical composition or a pharmaceutical formulation thereof can be administered to a subject at a dose of between about 0.05 μg/day to about 100 μg/day.

In certain embodiments, the kit can further include a device for administering the pharmaceutical composition. For example, but not by way of limitation, the device can include a syringe and/or contact lenses.

### EXAMPLES

The presently disclosed subject matter will be better understood by reference to the following Examples, which are provided as exemplary of the presently disclosed subject matter, and not by way of limitation.

### Example 1

Corneal stromal stem cells (CSSCs) are located in the corneal limbus close to the corneal limbal epithelial stem cells (Du et al., Stem Cells 23, 1266-1275 (2005); Funderburgh et al., Ocul Surf 14, 113-120 (2016)) and can be separated as a side population (Du et al., Stem Cells 23, 1266-1275 (2005)). These cells express stem cell markers CD90, CD73, CD105 and possess the ability to differentiate into cells of various lineages (Du et al., Stem Cells 23, 1266-1275 (2005); Kumar et al., Invest Ophthalmol Vis Sci 59, 3728-3738 (2018)). CSSC can suppress corneal scar formation in lumican knockout and Col3a1-EGFP genetic mouse corneal scar models (Du et al., Stem Cells 27, 1635-1642 (2009); Khandaker et al., Exp Eye Res 200, 108270 (2020)) and prevent corneal scar formation in an acute corneal wound model (Basu et al., Sci Transl Med 6, 266ra172 (2014); Weng et al., Eye Vis (Lond) 7, 52 (2020); Boote et al., Invest Ophthalmol Vis Sci 53, 2786-2795 (2012)). This finding opens the door for stem cell-based therapy for the treatment of corneal scars without corneal transplantation. Stem cell secretome, defined as the set of stem cell-secreted bioactive factors, including soluble proteins, growth factors, and extracellular vesicles, has regenerative effects (Willis et al., Front. Cell. Neurosci https://doi.org/10.3389/fncel.2020.590960 (2020)); Ranganath et al., Cell Stem Cell 10, 244-258 (2012)). Currently, sparse studies have investigated the effect of secretome on ocular regeneration and wound healing. The present Example assessed the therapeutic effect of CSSC secretome on a mouse corneal wound model and explored possible mechanisms.

### Methods

Cell culture and collection of secretome. Human corneal stromal stem cells (CSSCs) were obtained from donor corneas and cultured in Dulbecco's modified Eagle's medium (DMEM) low glucose mixed with different growth supplements (Du et al., Stem Cells 23, 1266-1275 (2005); Kumar et al., Invest Ophthalmol Vis Sci 59, 3728-3738 (2018)). Human corneal fibroblasts were cultured in DMEM/F12 plus 10% FBS as previously reported (Xiong et al., Elife 10 (2021); Du et al., Stem Cells 23, 1266-1275 (2005)). Culture media were replenished every third day. Secretomes from CSSC and fibroblasts were collected in the log phase as previously reported by culturing cells in a basal medium devoid of serum and any growth factors (Kumar et al., Invest Ophthalmol Vis Sci 59, 3728-3738 (2018); Kumar et al., Exp Eye Res 189, 107860 (2019); Kumar et al., Mol Neurobiol 54, 4672-4682 (2017); Kumar et al., Biochimie 155, 129-139 (2018); Kumar et al., Sci Rep 7, 15015 (2017)). Harvested secretomes from both CSSC and fibroblasts were further concentrated to 25X via centrifugation at 5,000 rpm in Amicon Ultra 100k cutoff ultrafilters. The concentrated secretome was mixed in a 1:1 ratio with 10 mg/ml fibrinogen and applied 1 μl to 0.5 μl of 100U/ml thrombin to form fibrin gel on the mouse corneas. The application can be repeated once.

Mouse corneal injury and secretome treatment. The experiments included four groups: 1) uninjured naïve normal controls; 2) mice received wound and fibroblast secretome; 3) mice received wound and CSSC secretome; and 4) mice received wound and sham (fibrinogen and thrombin to form fibrin gel without secretome). Twenty mice in each group were anesthetized by intraperitoneal injection of ketamine (50 mg/kg) and xylazine (5 mg/kg) and only one eye of each mouse was treated. One drop of proparacaine hydrochloride (0.5%) was added before debridement for topical anesthesia. A trephine was used to mark the central 2 mm of the cornea. An AlgerBrush II was passed over the arca marked by the trephine to perform corneal epithelial debridement. After the removal of the epithelium, the AlgerBrush was applied again to damage the basement membrane and the anterior stromal tissue as described previously (Boote et al., Invest Ophthalmol Vis Sci 53, 2786-2795 (2012)). Immediately after wound and sham or secretome treatment, mice received ketoprofen (3 mg/kg) for analgesia. 0.5 μl of thrombin (100 U/ml, Sigma) was added to the wounded cornea, followed by 1 μl of 1:1 secretome and fibrinogen (10 mg/ml, Sigma) mixture or fibrinogen only as sham control. After 1 minute, a second round of thrombin and fibrinogen was applied to the cornea. One drop of gentamicin ophthalmic solution (0.3%) was added to prevent bacterial infection.

Cell viability staining. Cells were stained with two viability dyes, Calcein Red-Orangeand Hoechst 33342 (Invitrogen) after secretome harvesting from both CSSC and fibroblasts to assess cell viability. Both cell types were washed once with phosphate-buffered saline (PBS) and incubated for 15 minutes in the dark with Calcein Red-Orange (1:1000) and Hocchst 33342 (1:2000). Staining was captured using 361 nm/565 nm excitation/emission wavelengths under an inverted fluorescent microscope (TE 200-E, Nikon).

Flow Cytometry. For stem cell characterization, cells were washed briefly with PBS, fixed and permeabilized wherever required (for nuclear and cytoplasmic antibodies), blocked in 1% bovine serum albumin (BSA) for an hour, and stained with the following antibodies: CD90-BV510, CD73-PE/Cy7, CD105-AF647, OCT4-FITC, CD166-FITC, ABCG2-APC, NOTCH1-PE, STRO1-AF647, and CD271-AF647. Isotype controls were used for the stem cell characterization experiment, IgG1 K Iso FITC, IgG2a K Isotype PE, IgG1 K Iso PE/CY7, IgG1 K Iso APC. At least 20,000 cells were acquired using FACS Aria and analyzed using FlowJo_V10 software. At least three different CSSC strains from 3 different donors were characterized for stem cell marker expression. For cell death and apoptosis analysis post secretome harvesting, cells were stained with annexin V for apoptosis and 7-AAD for necrosis in annexin V binding buffer using 30 minutes of incubation in dark. Cells were directly acquired after that using a flow cytometer. For flow cytometry analysis of inflammatory and immune cells, three days following treatment, corneas were examined and rinsed, minced, and treated with collagenase type I (84 U/cornea) for 60 min at 37° C., and triturated until no apparent tissue fragments remained. The single-cell suspension of each cornea was then filtered through a 40-μm cell strainer cap and washed. Corneal cells were treated with anti-mouse CD16/CD32 (Fc III/II receptor; 2.4G2) to prevent nonspecific antibody binding and then stained for various leukocyte surface markers for 30 min at 4° C. The following antibodies were used: CD45-PerCP, CD11b-AF700, GR1-PE, F4/80-PECy7, together with the Violet Live/Dead dye. The cells were analyzed via a Cytoflex cytometer and further analysis was carried out using FlowJo software. Gates were set based on staining with the appropriate single antibody and a mixture lacking that particular antibody, fluorescence minus one (FMO) control.

Optical Coherence Tomography (OCT) scanning and analysis. Mouse corneas were evaluated using optical coherence tomography (OCT) to examine the epithelium regeneration after the treatments. A modified Bioptigen spectral-domain OCT system (Bioptigen Inc., Durham, NC, and SuperLum Ltd., Ireland) was used to acquire image volumes of mouse corneas in vivo. Scans sampled a 3 by 3 by 3 mm region of tissue with 512 by 180 by 1,024 measurements. For each group, 12-20 eyes were examined by determining quadrant-scans along four axes (0 0-180 0, 45 0-225 0, 90 0-270 0, and 135 0-315 0) to ensure scanning through the central cornea and data along the 0 0 to 180 0axis was used for analysis. The OCT images were then evaluated via FIJI (NIH). For OCT analysis mice were grouped according to their epithelial healing: 1) healed (the cornea is comparable to that of mice with no wounding); 2) partially healed (slight opaqueness of the cornea/area outside of pupil not completely healed), or 3) not healed (the cornea is with ulcer/too thin/too thick). The data was then analyzed via chi-squared analysis at the alpha=0.05 level.

Immunofluorescence and Whole Mount Staining. For immunofluorescence staining, fixed corneas 12 μm cryosection were used. On each slide, 6-8 corneal sections were permeabilized using 0.1% triton-X for 15 minutes and blocked using 1% BSA for 1 hour. Cornea sections were incubated in primary antibodies overnight and secondary antibodies for 2 hours at room temperature using 4′,6-diamidino-2-phenylindole (DAPI) as nuclear staining. The primary antibodies used for the staining of cornea sections are as follows: CD45-eF450, F4/80-APC, GR1-PE, CD59-FITC, collagen IV. SPARC, α-SMA, collagen 3A1, vitronectin. After staining, slides were acquired using laser scanning confocal microscope (FV1200,Olympus) and analyzed using FV10-ASW4.2 Viewer (Olympus). Corneas were acquired from both central and peripheral positions and fluorescence intensity was averaged to give final mean fluorescence intensity (MFI) value using Image J (National Institute of Health). For whole-mount staining, mouse corneas were fixed for 1 hour in 1.3% paraformaldehyde in PBS at room temperature. Radial incisions were made to allow for flat mounting of the corneal tissues. Corneas were washed in PBS five times, permeabilized in 1% Triton-X-100 in PBS at room temperature for 60 minutes, and blocked with 20% goat serum in blocking buffer (0.3% Triton-X-100/0.1% Tween-20 in PBS) for 1 hour. The corneas were then incubated in a mixture of primary antibodies β-3 tubulin and P-substance for 2 hours at room temperature, followed by an additional incubation overnight at 4° C. After five 5-minute washes in a buffer (0.1% Tween-20 in PBS), the corneas were incubated in a mixture of following secondary antibodies in a blocking buffer at room temperature for 2 hours. Following five 10-minute washes with a wash buffer, the corneas were mounted on slides and dried at 4° C. for at least 12 hours before imaging. Images were acquired in multiple z-stacks by sequential scanning on an FV1200 confocal microscope and stitched on FV10 viewer. The following secondary antibodies were used for immunofluorescence and wholemount staining: Donkey anti-goat IgG AF-555, Donkey anti-rabbit AF-555 IgG, Donkey anti-mouse AF-647 IgG, Donkey anti-Rabbit AF-488 IgG, and Donkey anti-rat-488 IgG. For IgG autoantibody staining, Goat anti-mouse IgG-647 was used. The immunofluorescent experiments were performed at least in three different corneas per group and repeated 2-3 times. Mean fluorescence intensity quantifications were done by two independent observers.

TUNEL staining. Corneal cell death detection using TUNEL staining was performed using a commercially available kit, as per the manufacturer's instructions on cryosections of corneal tissue. Nuclei were stained with DAPI. At least three independent eyes from each condition and eight sections of each condition were stained and imaged using a confocal microscope. For quantification, TUNEL+ cells were counted manually by two independent observers.

Blinded methods were used for study outcome and designs. In particular, the microscopy and histological evaluations of cornea section immunofluorescence or whole mount staining for different antibodies was performed by two independent researchers. Flow cytometry was performed and analyzed by two independent researchers. OCT was performed and analyzed by a researcher in blinded fashion. The animal experiments were repeated at least twice and secretome from at least two different CSSC and fibroblasts were used for the experiment.

Multidimensional protein identification technology (MudPIT) analysis. Proteomic identification and analysis of the secretome were performed for both CSSC and fibroblasts. Secretome was harvested and concentrated as described above. The secretome proteins were precipitated using methanol chloroform method of Wessel and Flugge (Wessel et al., Anal Biochem 138, 141-143 (1984)). These proteins were processed through various steps of denaturation, reduction, alkylation, and digestion as reported before (Kumar et al., Biorxiv 10.1101/2021.06.18.449038 (2021)). Briefly, peptides obtained after the process were identified using LTQ orbitrap Elite mass spectrometer equipped with a nano-LC electrospray ionization source (ThermoScientific). Tandem mass (MS/MS) spectra were interpreted using ProluCID v. 1.3.3. DTASelect v 1.9 (Tabb et al., J Proteome Res 1, 21-26 (2002)) and swallow v. 0.0.1 (https://github.com/tzw-wen/kite) were used to filter ProLuCID search results at estimated false discovery rates (FDRs) at the spectrum, peptide, and protein levels. All reported FDRs are less than 5%. All 4 data sets were contrasted using Contrast v 1.9 against their merged data set, using the software sandmartin v 0.0.1. (Zhang et al., Anal Chem 82, 2272-2281 (2010)). NSAF7 v 0.0.1 was used to generate spectral count-based label-free quantitation results.

GO enrichment analysis was performed using DAVID (version DAVID 6.8;http://david.ncifcrf.gov/) and functional enrichment of the genes whose corresponding proteins have positively distributed normalized spectral abundance factor (dNSAF) were analyzed using GOstats (version 2.48.0) in both replicates of CSSC and fibroblasts. The top 10 most significantly enriched GO categories for a given cell type were compared.

R package pheatmap (version 1.0.12) was used for hierarchical clustering analysis and heatmap plotting. Heatmaps were used to compare the protein expression in the secretome of CSSC and fibroblasts using dNSAF protein values. In the heatmap, row represents the name of the heatmap, and columns indicate the cell type used. Similar elements were classified in groups in a binary tree using hierarchical clustering.

Statistical analysis. All data reported in the present Example is shown as mean ±SD. Statistical differences were determined using one-way analysis of variance to assess the significance of differences between all groups and reported as significant p values for multiple comparisons and adjusted by the Tukey method. Statistical significance was set at p<0.05.

### Results

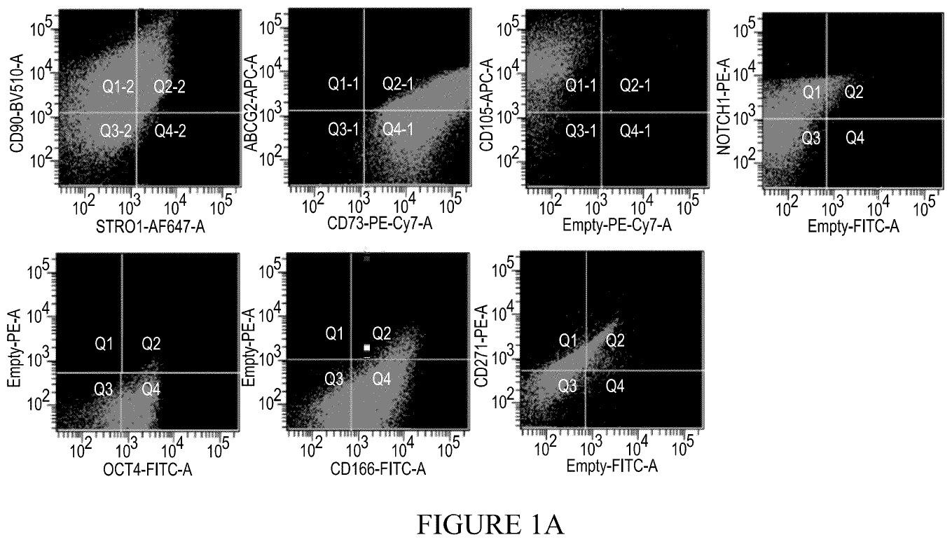

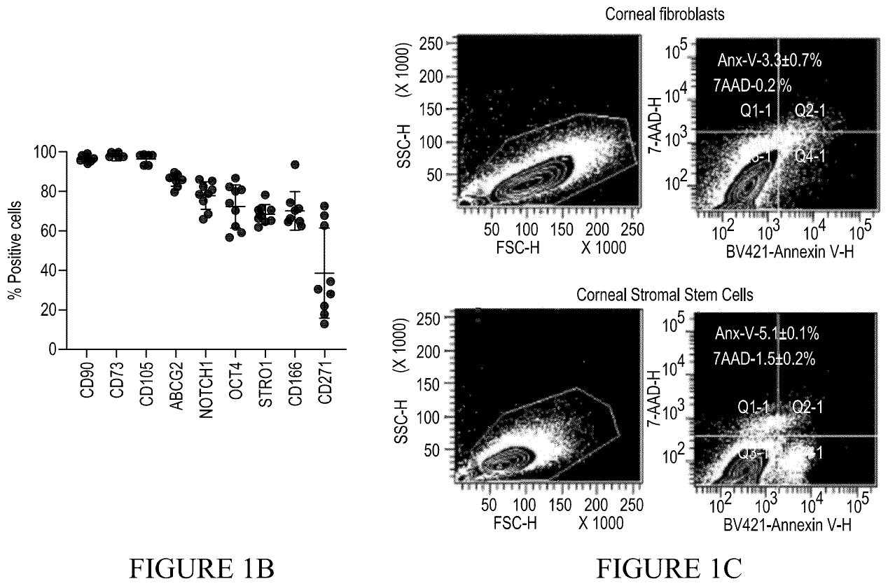

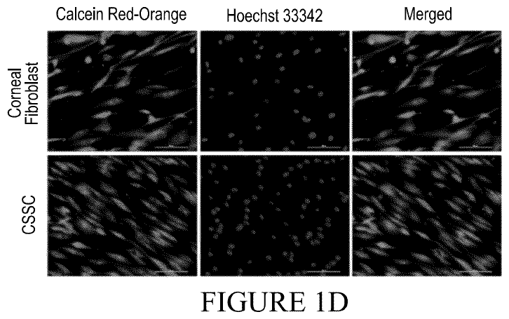

Corneal stromal stem cell secretome promotes scarless corneal wound healing and dampens inflammation. Human corneal stromal stem cells (CSSC) were characterized for their stem cell properties with the expression of stem cell markers CD90, CD73, CD105 as per the guidelines of International Society of Cell Therapy (Kumar et al., Invest Ophthalmol Vis Sci 59, 3728-3738 (2018); Dominici et al., Cytotherapy 8, 315-317 (2006)) and with additional stem cell markers OCT4, CD166, NOTCH1, STRO1, and ABCG2. CSSC expressed>90% CD90, CD73 and CD105, 70-80% ABCG2, NOTCH1, OCT4, STRO1 and CD166, and ˜40% CD271 (FIGS. 1A and 1B). This phenotype reflected the stem cell nature of CSSC. Corneal fibroblasts were cultured and characterized with negative expression to the stem cell markers and expression of fibronectin and without expression of the stem cell markers as previously described (Xiong et al., Elife 10 (2021); Du et al., Stem Cells 23, 1266-1275 (2005)). Secretomes from both CSSC and corneal fibroblasts were collected after they were cultured in serum-free conditions for 48 hrs. The cell viability of both CSSC and corneal fibroblasts post secretome harvesting was evaluated by Annexin V/7-Aminoactinomycin D (7-AAD) staining using flow cytometry and live-cell stain with viability dyes Calcein Red-Orange and Hoechst 33342. Flow cytometry results showed 3.3±0.7% apoptosis in corneal fibroblasts and 5.1±0.1% apoptosis in CSSC (FIG. 1C) which had no statistical significance. No cell death was observed after Calcein Red-Orange and Hoechst 33342 staining in either corneal fibroblasts or CSSC (FIG. 1D), demonstrating that secretomes were harvested from healthy cells.

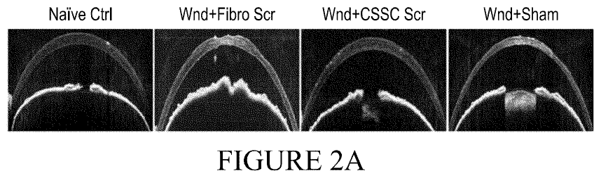

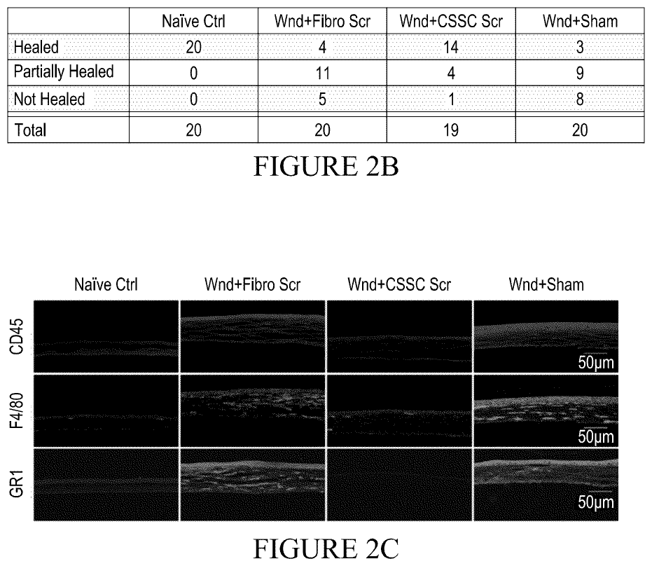

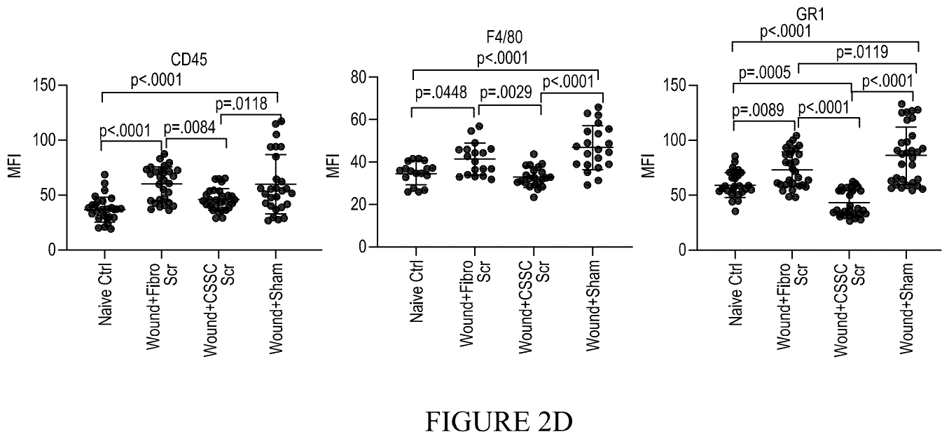

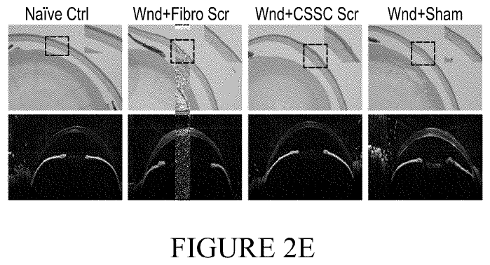

The harvested secretome was then examined to determine whether it induced regeneration in wounded mouse corneas. A corneal wound removing the corneal epithelium, Bowman's member, and superficial stroma was induced using an Algerbrush II as previously described (Basu et al., Sci Transl Med 6, 266ra172 (2014); Weng et al., Eye Vis (Lond) 7, 52 (2020); Boote et al., Invest Ophthalmol Vis Sci 53, 2786-2795 (2012); Shojaati et al., Stem Cells Transl Med 7, 487-494 (2018)). Immediately after the wound, 0.5 μl of 100 U/ml thrombin was applied to the wound site, and 1 μl of 1:1 mixture of 10 mg/ml fibrinogen and 25× concentrated secretome (either from CSSC or fibroblasts) or medium as a sham control was added to the thrombin on top of the corneal wound to form fibrin gel attaching to the wounded corneal surface. After a minute, a second round of thrombin and mixed fibrinogen and secretome was applied. The corneas were examined 72 hours later. Optical coherence tomography (OCT) was used to produce high-resolution cross-sectional images of the cornea located in the anterior segment of the eye (Basu et al., Sci Transl Med 6, 266ra172 (2014); Venkateswaran et al., Eye Vis (Lond) 5, 13 (2018)). OCT scanning images showed that the corneas that received sham treatment and receiving fibroblast secretome treatment showed corneal scar formation suggested by the higher pixels in the images. The CSSC secretome treatment group showed an intact epithelial layer and transparent stroma without signs of scar formation comparable to the corneas of naïve control mice (FIG. 2A). The corneas were classified as healed, partially healed, and not healed based on the OCT images. CSSC secretome healed 73.6% of corneas (14 out of 19 corneas) and partially healed 21% of corneas (4 out of 19 corneas). Only 5.2% of corneas remained non-healed (1 out of 19 corneas) (FIG. 2B). In contrast, fibroblast secretome healed only 20% of corneas fully (4 out of 20 corneas) and 52.3% of corneas partially (11 out of 20 corneas). Here, 25% of corneas (5 out of 20 corneas) remained non-healed, which was comparable to sham controls. Sham controls healed 15% of corneas (3 out of 20 corncas) and partially healed 45% of corneas (9 out of 20 corneas). For the sham controls, 40% of corneas remained non-healed (8 out of 20 corneas). FIG. 2E confirms the results observed at day 3 after treatment (FIG. 2A-2D) and demonstrated that wounded corneas have fully healed by day 7, further supporting the positive impact of the CSSC secretome in preventing corneal scar formation.

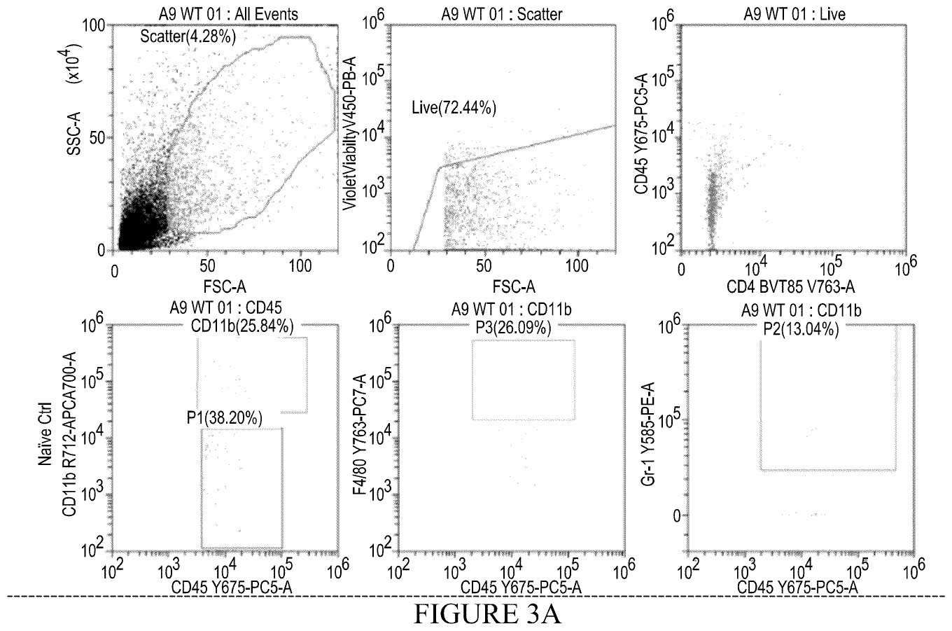

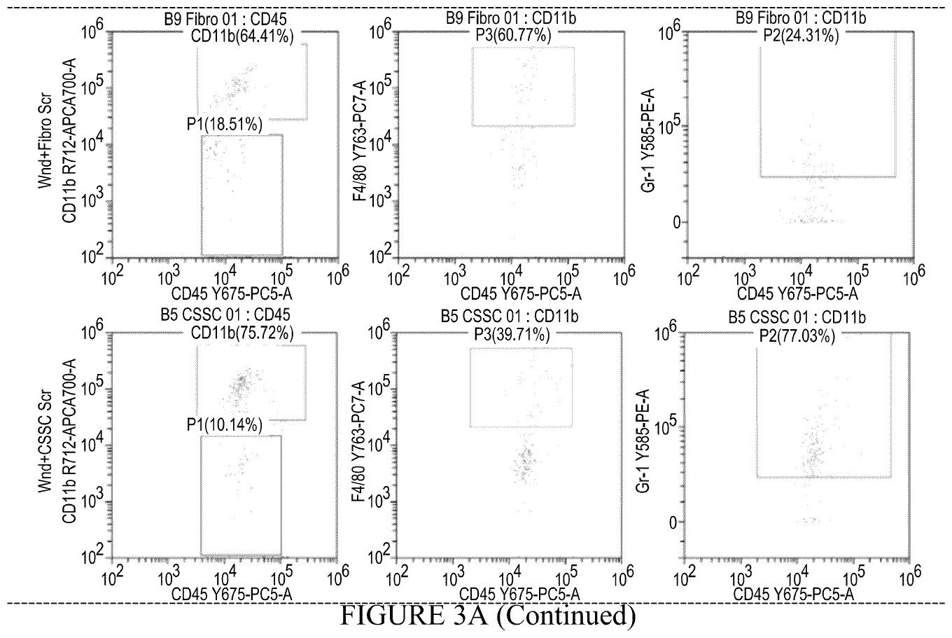

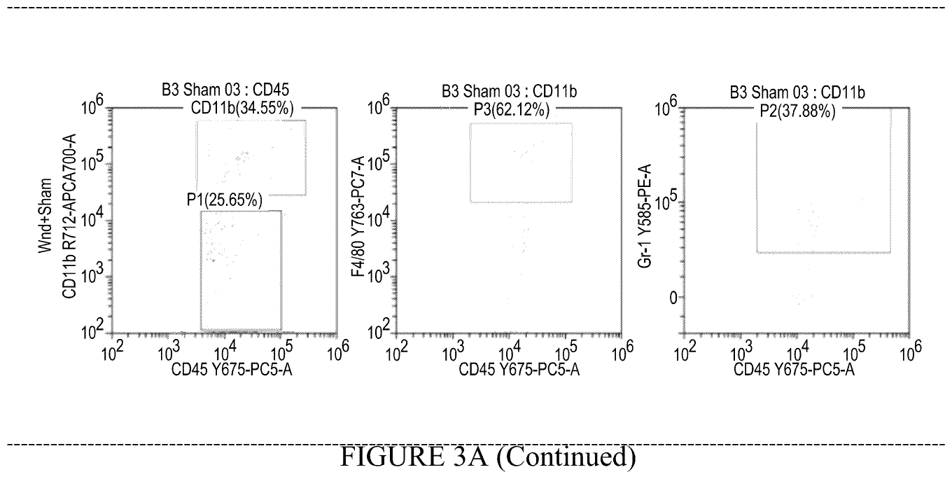

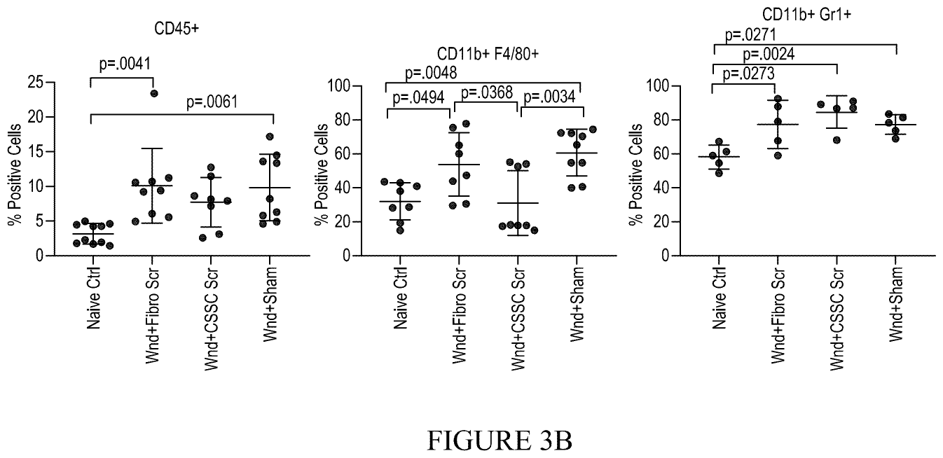

Inflammation plays a critical role in many cellular processes. In vascular tissues, it helps to clear pathogen by increasing vascular cell availability to the wounded arca, but it can be a liability in an avascular tissue like the cornea and aggravate corneal scarring and damage. The cornea is an immune privileged site, partly due to its avascular nature. Inflammation in the cornea can lead to compromised corneal integrity and promote cell death by infiltration of inflammatory and immune cells, leading to deposition of fibrotic ECM, neovascularization, and tissue destruction (Josephson et al., J Am Optom Assoc 59, 679-685 (1988)). CSSC have been shown to have anti-inflammatory effects via TSG-6 activation (Hertsenberg et al., PLOS One 12, e0171712 (2017)). Thus, the present Example also assessed whether CSSC secretome has similar effects to reduce inflammation. CD45 is a commonly used marker for hematopoietic cells except for red blood cells and platelets and its increase is associated with the activation of inflammatory cells (Kumar et al., Prog Retin Eye Res 10.1016/j.preteyeres.2021.101011, 101011 (2021); Zhang et al., Proc Natl Acad Sci U S A 118 (2021)). CD45 expression increased in the wounded mouse corneas with sham treatment (59.9±27.1) and fibroblast secretome treatment (60.1±15.5). However, CD45 expression reduced significantly after CSSC secretome treatment (45.8±9.7), comparable to naïve control corneas (36.9±11.2) measured as mean fluorescent intensity (MFI) by immunofluorescent staining (FIGS. 2C and 2D). Immune cell infiltration in the corneas was assessed by immunostaining to detect the expression of F4/80, a well-characterized macrophage marker expressed at a high level in many macrophages like Kupffer cells and microglia. CD45+/F4/80+ cells have been reported to increase after corneal injury (Pal-Ghosh et al., Invest Ophthalmol Vis Sci 55, 2757-2765 (2014)). As shown in FIGS. 2C and 2D, F4/80 expression significantly increased after corneal wound with sham treatment (46.8±10.4) as compared to naïve control (34.8±5.3). However, F4/80 expression significantly reduced after CSSC secretome treatment (32.9±4.7). Fibroblast secretome did not reduce F4/80 expression (41.4±7.5). Another macrophage/myeloid marker Gr1, which stains two Ly6 family Glycosylphosphatidylinositol (GPI) anchor proteins Ly6G (granulocyte marker) and Ly6C (macrophage marker) (Ribechini et al., Eur J Immunol 39, 3538-3551 (2009); Dunay et al., Immunity 29, 306-317 (2008)), was also evaluated. Similarly, Gr1 was significantly increased after corncal wound (91.8±29.6) and reduced after CSSC secretome treatment (42.8±11.7) (FIGS. 2C and 2D). These results indicate that CSSC secretome, not fibroblast secretome, can promote corneal wound healing by dampening inflammation. The expression of CD45+ (inflammatory cells), CD11b+F4/80+ (dendritic cells), and CD11b+Gr1+ (neutrophils) was further analyzed by flow cytometry and showed similar results with increased different types of inflammatory cells after corneal wound and with fibroblast secretome treatment, but CSSC secretome reduced CD45+ and CD11b+F4/80+ cell numbers, comparable to naïve control animals (FIGS. 3A and 3B), although CSSC secretome did not reduce the expression of CD11b+Gr1+ cells. The results indicate that CSSC secretome promotes wound healing and dampens inflammation by preventing infiltration of inflammatory cells on the wounded cornea.

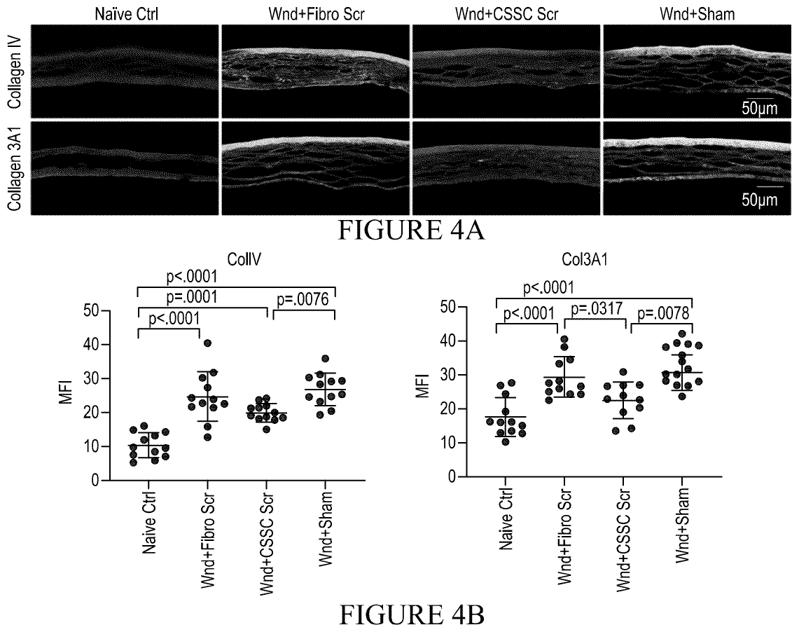

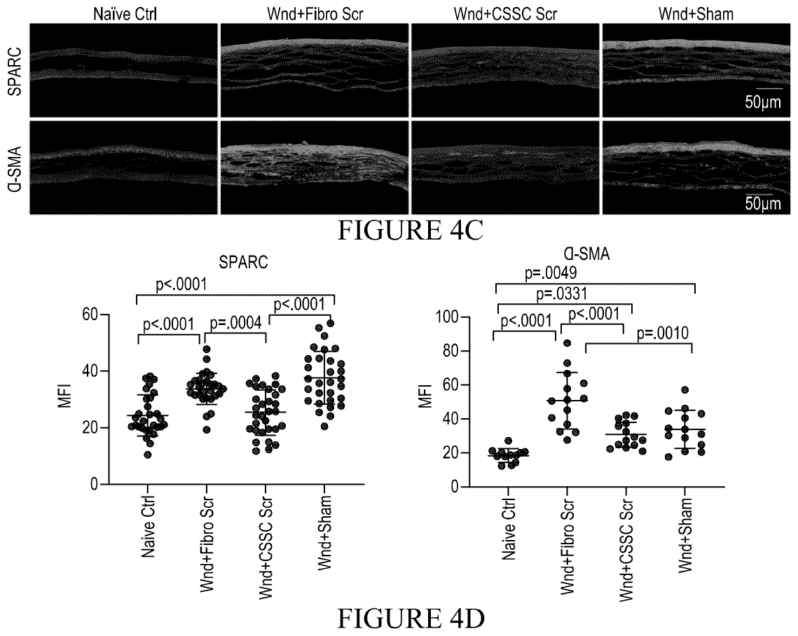

Stem cell secretome reduces fibrotic extracellular matrix deposition after corneal wound. During corneal wound healing, corneal keratocytes differentiate into fibroblasts and myofibroblasts which secret fibrotic extracellular matrix (ECM) to promote wound healing as well as fibrotic scar formation (Jester et al., Invest Ophthalmol Vis Sci 35, 730-743 (1994); Funderburgh et al., J Biol Chem 278, 45629-45637 (2003)). To assess whether CSSC secretome alters the ECM deposition and fibrosis during corneal wound healing, the expression of collagen IV (ColIV), collagen 3A1 (Col3A1), and α smooth muscle actin (α-SMA) in the corneas was monitored. ColIV and fibronectin are associated with fibroblastic cells in the injured cornea and corneal scar formation (Ishizaki et al., Curr Eye Res 16, 339-348 (1997)). ColIV expression was increased by almost 2.5-fold in the scarred corneas treated with sham (26.9±4.8) and with fibroblast secretome (24.8±7.3), as compared to naïve control corneas (10.5±3.6). After application of CSSC secretome, however, ColIV expression reduced significantly (20.1±2.6) (FIGS. 4A-4D). Col3A1 is highly expressed in scarred cornea and is widely used as a marker of corneal stromal fibrosis (Khandaker et al., Exp Eye Res 200, 108270 (2020)). CSSC secretome reduced the Col3Al expression (22.5±5.4) which was increased in the wounded corneas with sham treatment (30.6±5.1) and with fibroblast secretome treatment (29.2±5.9) (FIGS. 4A-4D).

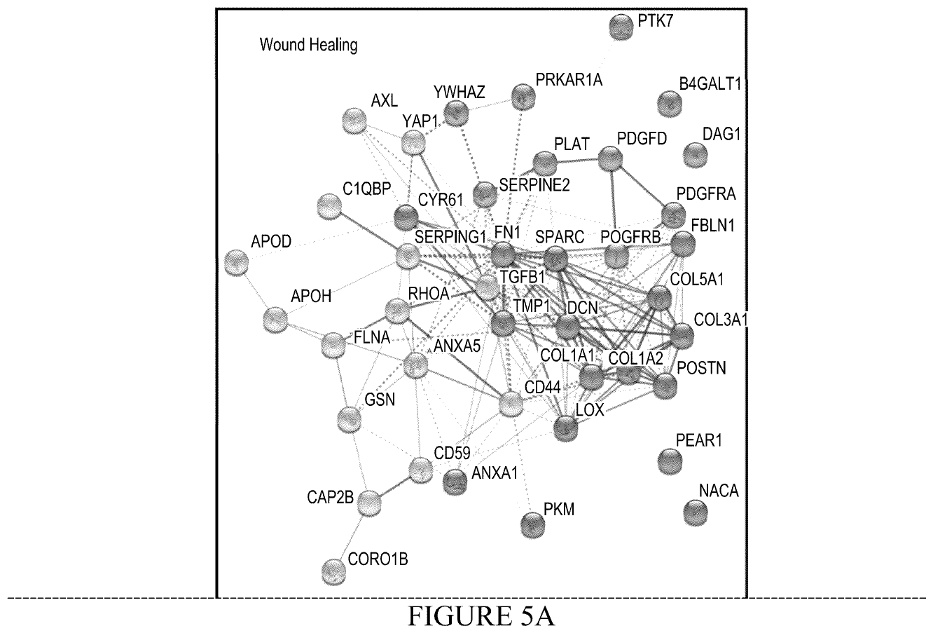

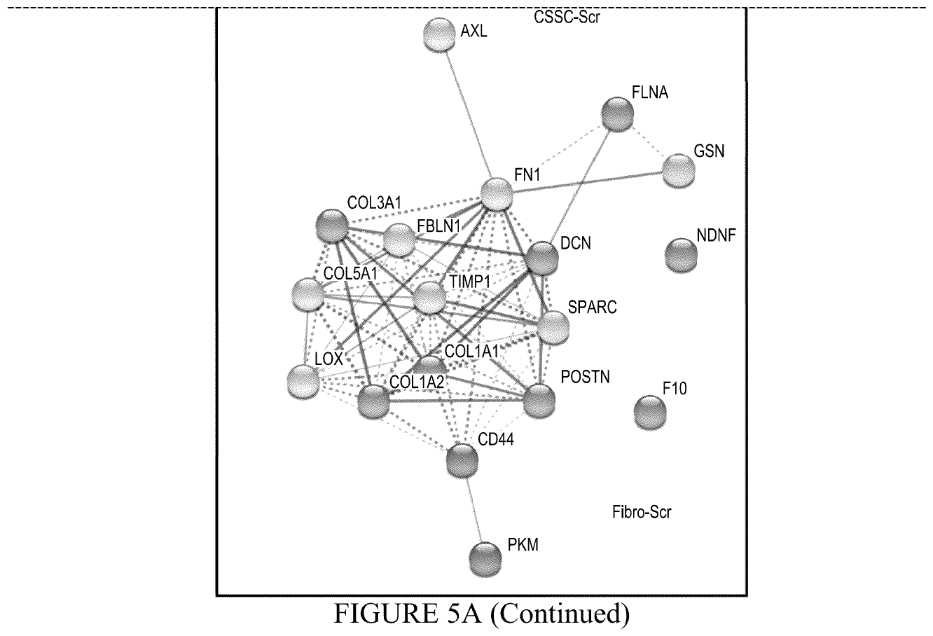

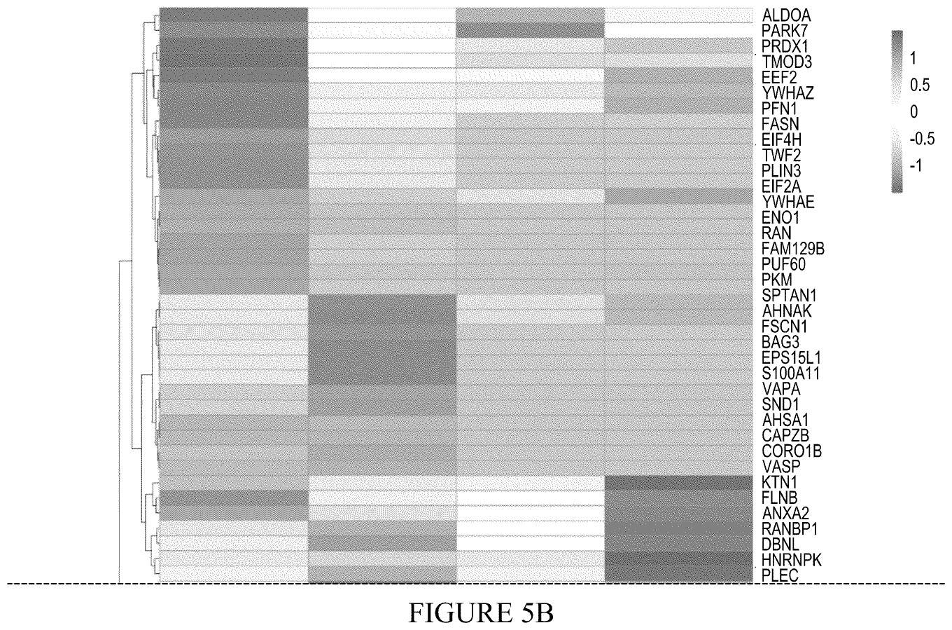

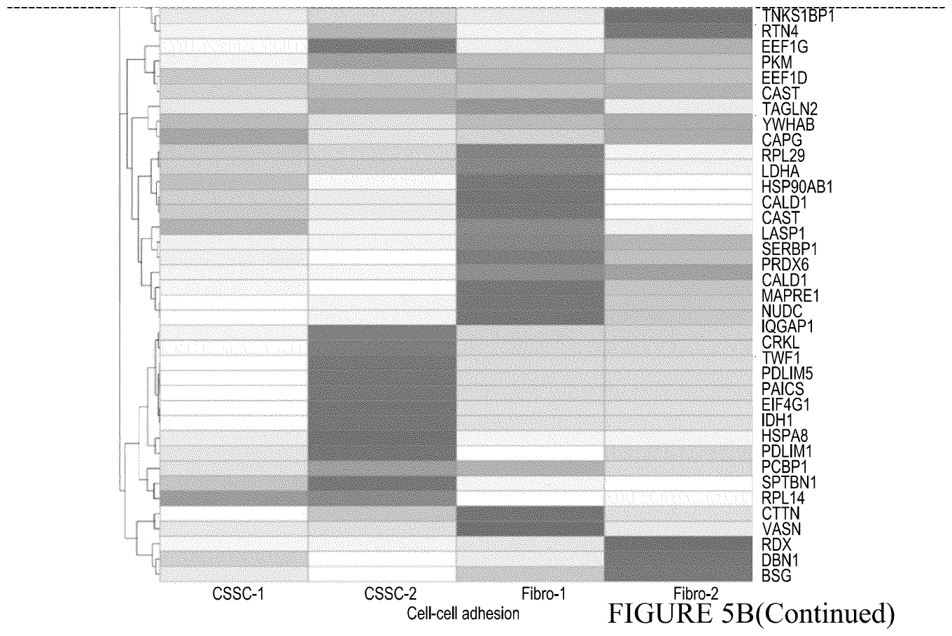

Secreted protein acidic and rich in cysteine (SPARC) is a collagen-binding protein and binds to collagen III and IV by its E-C domain to facilitate their assembly to ECM (Trombetta-Esilva et al., Open Rheumatol J 6, 146-155 (2012); Sasaki et al., EMBO J 17, 1625-1634 (1998)). SPARC expression is increased during corneal fibrosis as shown previously (Kumar et al., Invest Ophthalmol Vis 59, 3728-3738 (2018); Funderburgh et al., J Biol Chem 278, 45629-45637 (2003); Kumar et al., Exp Eye Res 189, 107860 (2019)). SPARC expression was significantly increased in the wounded corneas treated with sham (37.6±9.3) and with fibroblast secretome (33.8±5.5), as compared to naïve control corneas (24.4±7.3). CSSC secretome treatment reduced SPARC expression significantly (25.2±8.0) (FIGS. 4A-4D). α-SMA is expressed by activated fibroblasts and myofibroblasts in wounded corneas (Karamichos et al., Invest Ophthalmol Vis Sci 51, 1382-1388 (2010)). The expression of α-SMA was almost doubled after corneal wound with sham treatment (34.0±11.3) as compared to control (18.6±4.1) (FIGS. 4A-4D). Interestingly, fibroblast secretome worsened the condition by increasing the expression of α-SMA almost three-fold (51.0±16.6). CSSC secretome (30.9±7.5) did not induce any significant reduction of α-SMA as compared to the sham control. This finding emphasizes that CSSC secretome does induce regeneration in wounded corneas by reducing the expression of fibrotic markers and fibrotic ECM deposition. Comparative label-free protein identification and quantification (proteomics) by liquid chromatography coupled tandem mass spectrometry (LC-MS/MS) was performed on the secretomes from CSSC and corneal fibroblasts. Interestingly, CSSC secretome was found to contain 41 proteins that promote corneal wound healing and fibroblast secretome only contained 18 such proteins related to corneal wound healing (FIG. 5A). CSSC secretome was also found to contain a high number of cell-cell adhesion proteins or molecules while fibroblast secretome contains fewer such proteins (FIG. 5B). These data indicate that these cell-cell adhesion proteins can contribute to the scarless wounded healing promoted by CSSC secretome.

Stem cell secretome promotes corneal sensory nerve regeneration. One of the major complications resulting in vision loss in the corneal wound is the loss of sensory neurons. Infiltration of immune cells in the cornea usually causes death of sensory neurons (Kumar et al., Prog Retin Eye Res 10.1016/j.preteyeres.2021.101011, 101011 (2021); Chucair-Elliott et al., Invest Ophthalmol Vis Sci 58, 4670-4682 (2017)). Blocking this interaction might prevent corneal opacity (Yun et al., Immunity 53, 1050¬1062 e1055 (2020)). Death of sensory neurons can lead to loss of blinking response which can inhibit tear production and distribution. This can result in dry eye due to corneal desiccation and aggravation of corneal inflammation and scarring. Since CSSC secretome can prevent inflammatory cell infiltration and dampen inflammation, it might be able to protect sensory nerves.

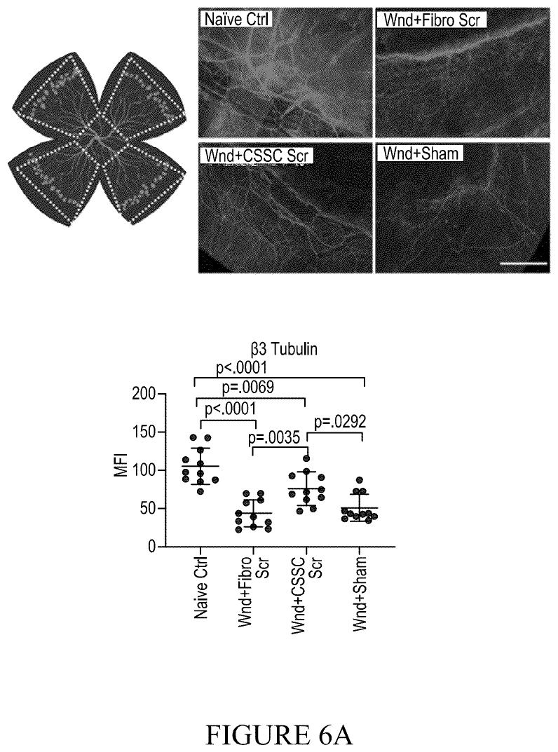

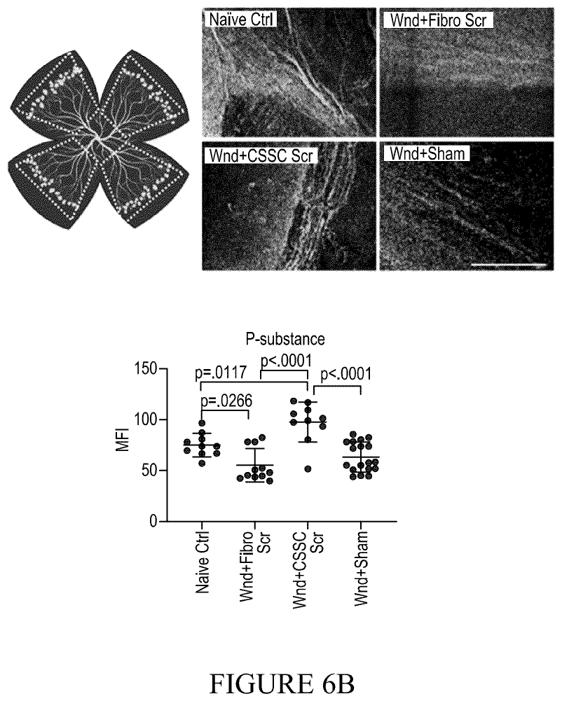

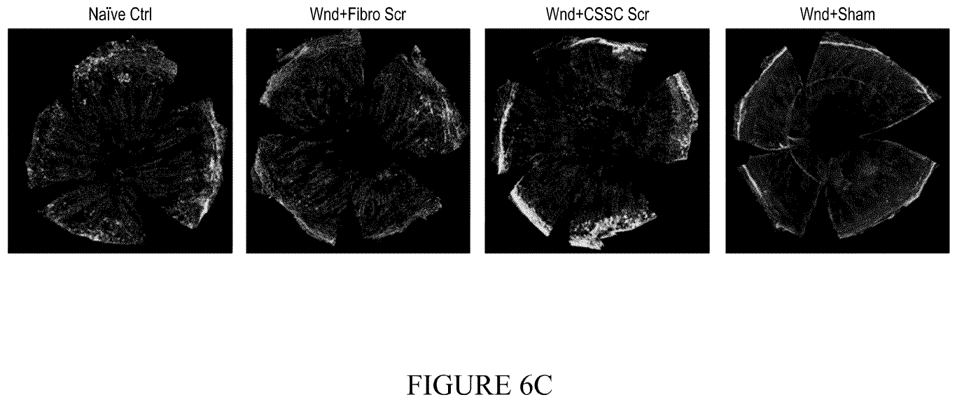

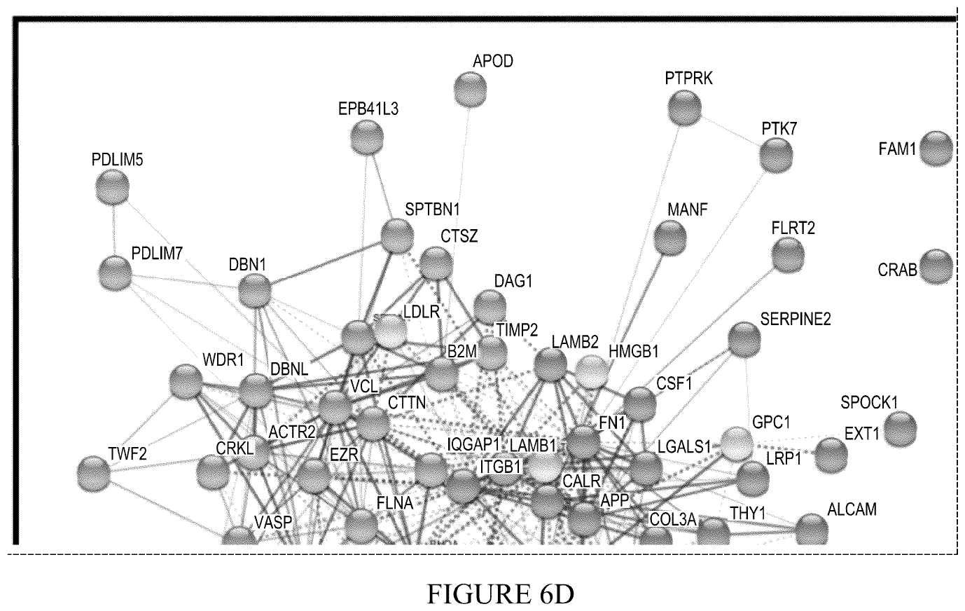

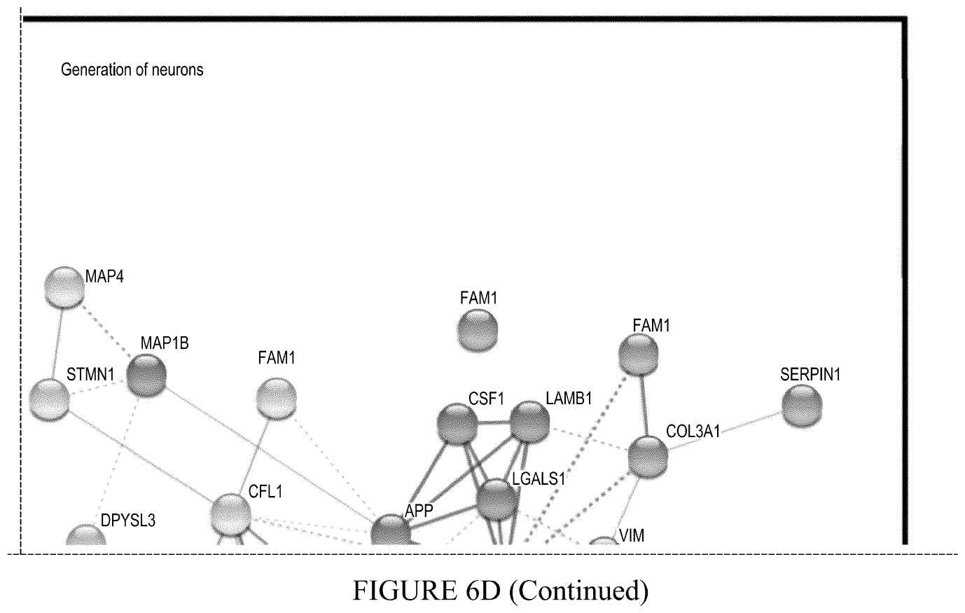

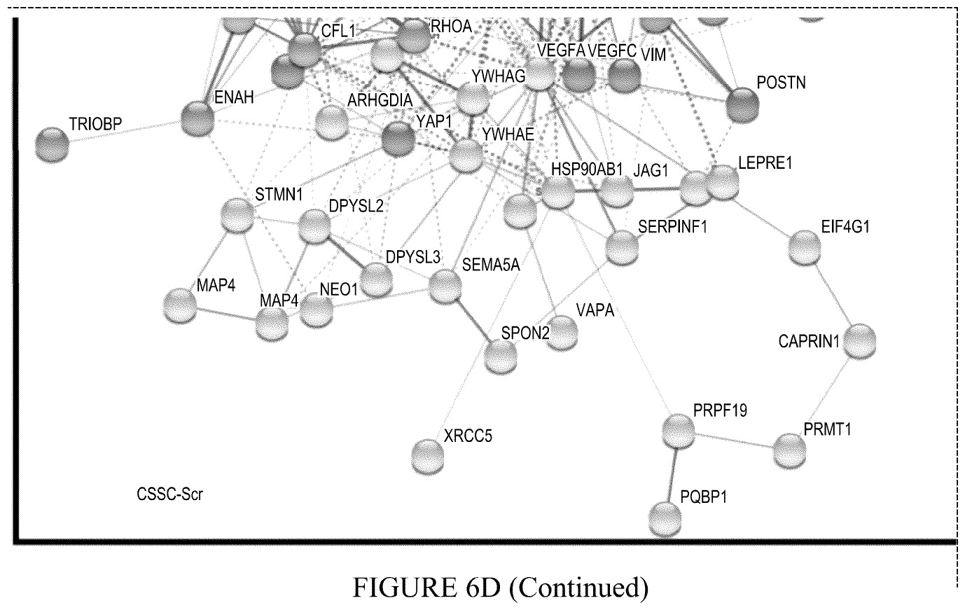

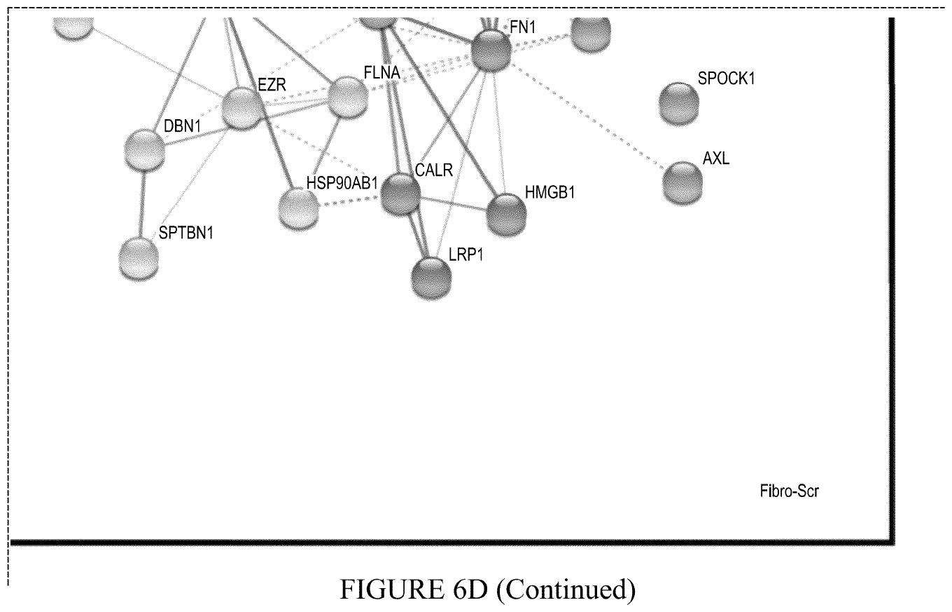

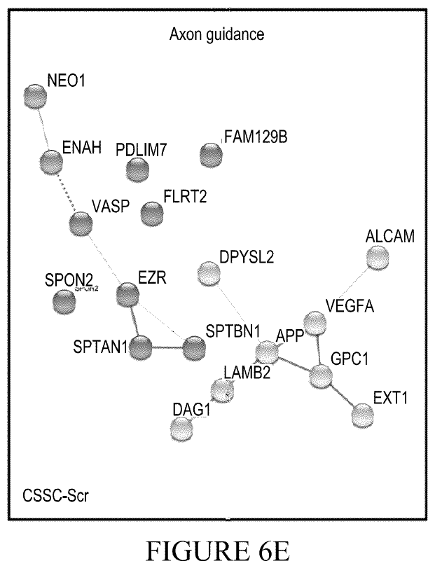

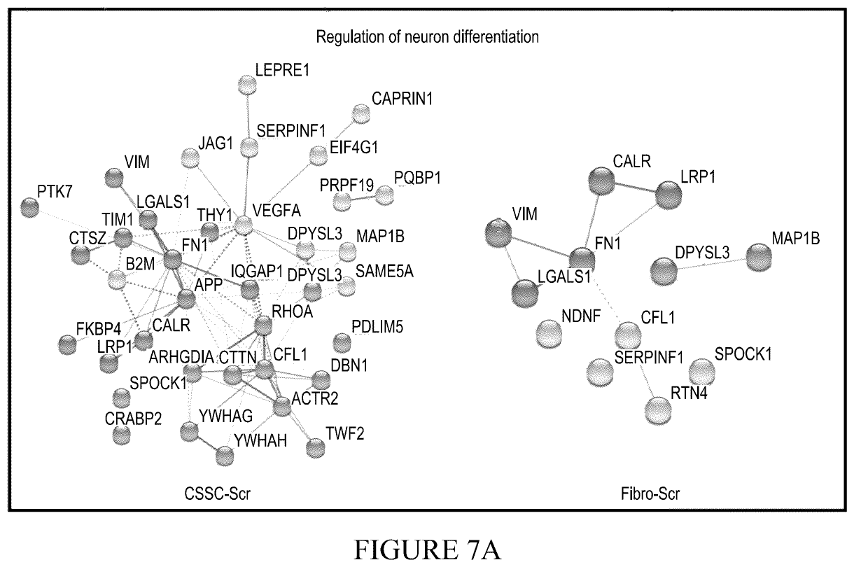

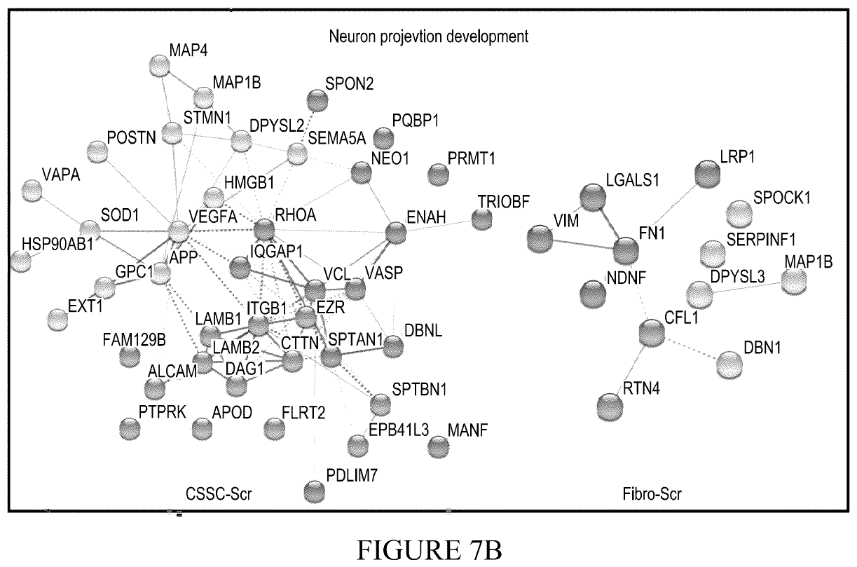

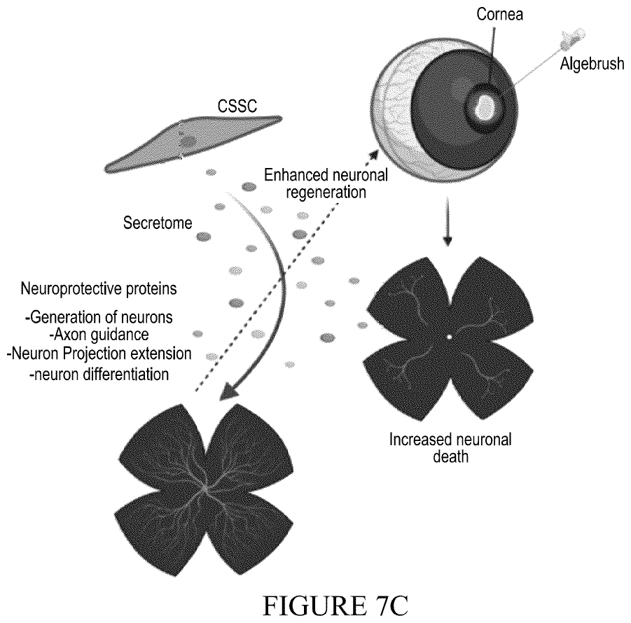

Two neuronal markers β-3 tubulin and P-substance were examined in the whole mount corneas by immunostaining. Overall, naïve control corneas showed plenty of sensory nerve fibers in the cornea with positive β-3 tubulin staining (106.1±23.6, FIG. 6A). The wounded corneas that received CSSC secretome had partially regenerated sensory nerve plexus (76.2±21.4), whereas fibroblast secretome (44.2±17.8) and sham treatment groups (51.1±11.4) showed much less sensory nerve fibers (FIG. 6A). Similarly, the expression of P-substance, a sensory neuron marker, was significantly increased in the wounded mouse corneas that received CSSC secretome treatment (97.06±19.6) in comparison to that treated with sham (62.99±14.4) and with fibroblast secretome (54.81±16.3), which were reduced as compared to the naïve control (74.61±11.41) (FIG. 6B). FIGS. 6A-6C confirms the CSSC secretome effect on the preservation of corneal sensory nerves observed at day 3 after treatment (FIG. 6A-6B) and demonstrated that at day 7 the axons extend to the central corneas in CSSC Scr treatment, similar to the naïve control, while the axons didn't extend to the central region in the corneas treated with Fibro Scr and Sham (FIG. 6C). Proteomic data show that 82 different proteins in CSSC secretome pertain generation of neurons such as TIMP1, TIMP2, SEMA5A, while in fibroblast secretome, only 26 of such proteins were identified (FIG. 6D). Axon guidance proteins guide axons during development so that neurons can navigate to their specific targets in a complex microenvironment. In CSSC secretome, 18 axon guidance proteins were found but none in fibroblast secretome (FIG. 6E). Further protcomic analysis showed more neural differentiation proteins and proteins involved in the development of neuron projections as compared to fibroblast secretome (FIG. 7A and 7B). Cumulatively, the proteins involved in wound healing, generation of neurons, axon guidance, neuron projection development, and neuron differentiation might enhance the overall therapeutic effect of CSSC secretome on corneal wound healing and neuron regeneration (FIG. 7C).

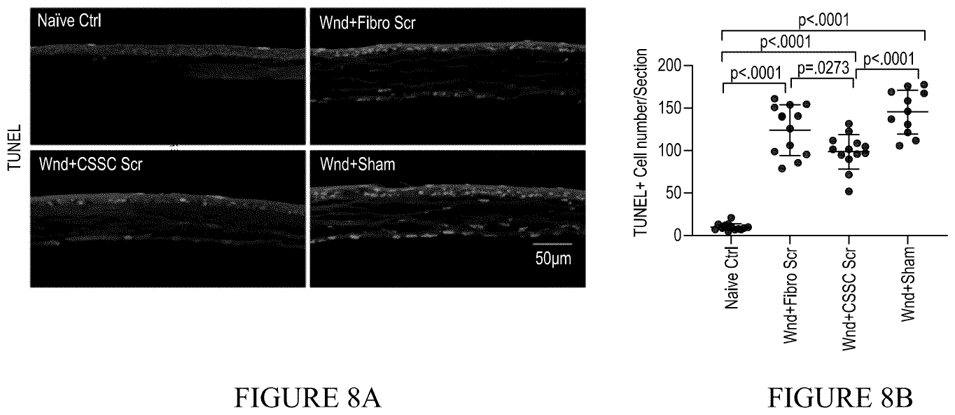

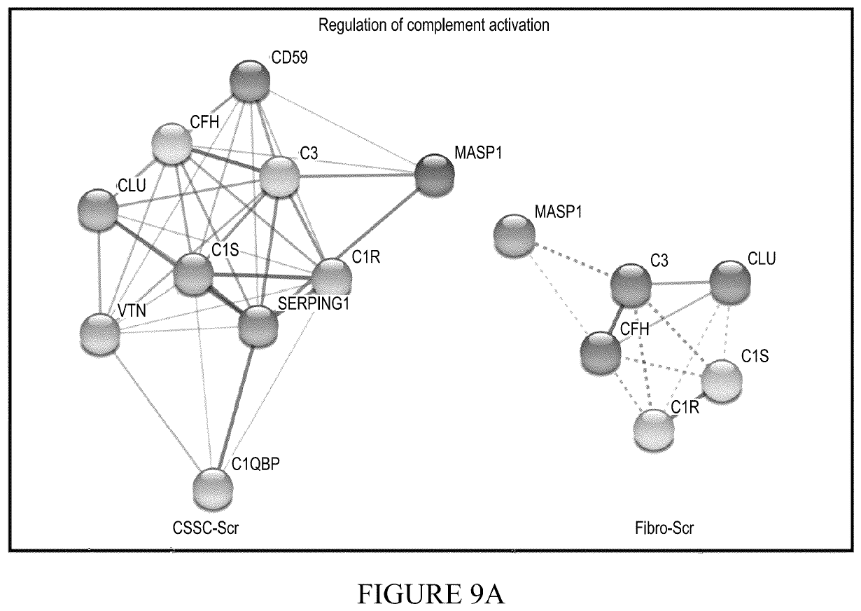

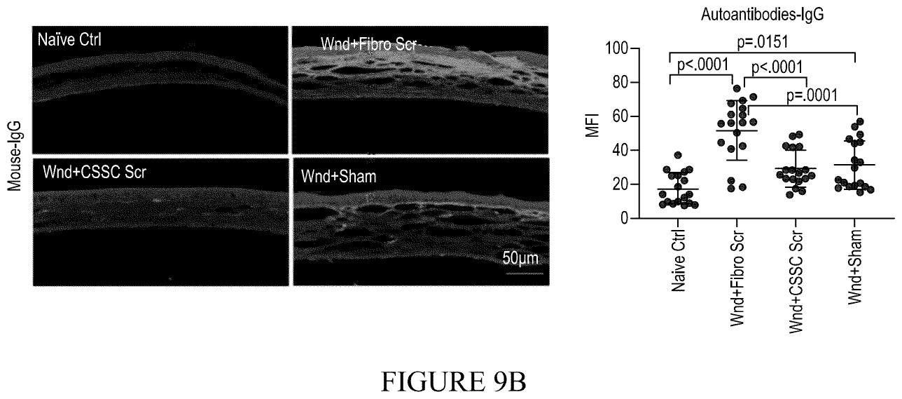

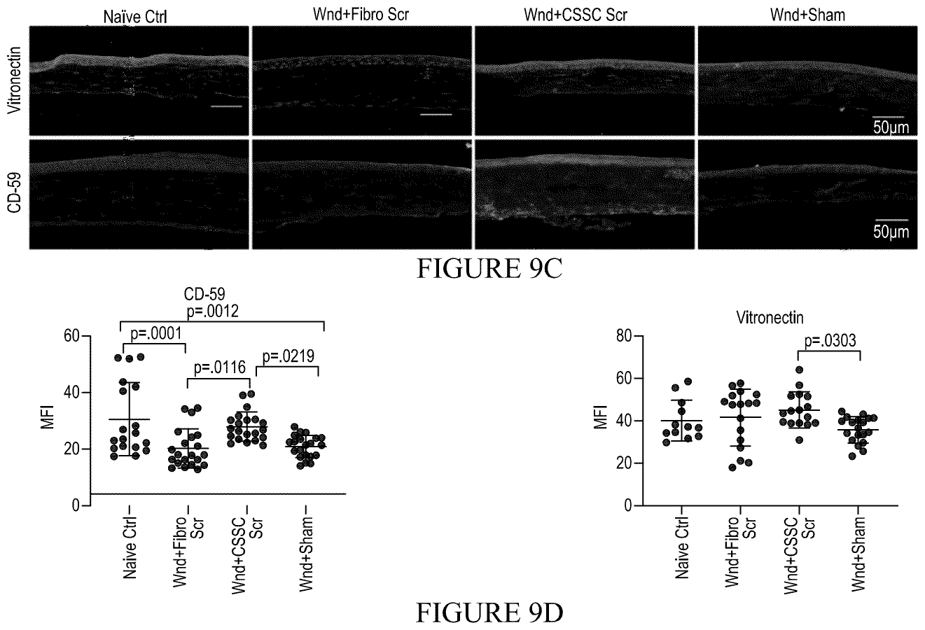

Stem cell secretome rescues corneal cell death by inhibiting complement system. Corneal wounds induce a massive cell death in the cornea by promoting inflammation and apoptosis (Ljubimov et al., Prog Retin Eye Res 49, 17-45 (2015)). To detect if CSSC secretome can protect corneal cells from death in wounded corneas, terminal deoxynucleotidyl transferase dUTP nick end labeling (TUNEL) staining was performed on the cryosectioned corneal tissue to detect DNA fragmentation by labeling the 3′-hydroxyl termini in the double-strand DNA breaks in apoptotic cells. There was approximately 15-fold higher cell death in wounded corneas treated with sham (146.0±25.9) as compared to naïve control corneas (10.3±4.2) (FIG. 8A and 8B). CSSC secretome treatment (98.9±20.3) significantly reduced the number of TUNEL+ cells undergoing apoptosis, while fibroblast secretome did not reduce the TUNEL+ cell number significantly (124.5±29.7). Since wounded corneas have more inflammatory cells and generally, inflammation is mediated by activated complements, it was questioned whether there is any involvement of complements in corneal wound healing by CSSC secretome. The proteomic analysis revealed a remarkable difference in the complement-related proteins in CSSC and fibroblast secretomes (FIG. 9A). CSSC secretome expressed four unique complement proteins CD59, SERPING1, C1QBP, and vitronectin while fibroblast secretome had no expression on any of these proteins (FIG. 9A). Surprisingly, all these four proteins inhibit complements. Complements usually trigger cell death by the formation of membrane attaching complex (MAC) and caspase activation. The formation of MAC was evaluated by detecting the autoantibodies stained with anti-mouse IgG antibodies. Naïve control corneas showed minimal autoantibody formation on the corneal cells (17.3±9.3) which was significantly increased in wounded corneas treated with sham (31.2±14.5, p=0.0151). CSSC secretome treatment reduced the level of autoantibodies (29.0±10.9) comparable to the Fibroblast secretome treatment dramatically increased the naïve control (p=0.0521). autoantibody level (51.7±17.7, p<0.0001) (FIG. 9B). This led us to investigate the involvement of proteins regulating complements in the secretomes. Two such proteins CD59and VTN in the mouse corneas were examined by immunostaining. CD59 expression was significantly reduced in wounded corneas treated with sham (20.8±3.9) and with fibroblast secretome (20.2±6.9) as compared to naive control (30.4±12.9), which was significantly increased (27.8±5.2) after CSSC secretome treatment, comparable to the level in naïve control (p=0.7205) (FIG. 9C). Interestingly, vitronectin expression was not altered significantly in wounded corneas after treatment with sham (35.9±6.2) or fibroblast secretome (41.6±13.4) as compared to naive control (40.2±9.6). CSSC secretome treatment on the wounded corneas significantly increased the vitronectin expression (45.2±8.4) as compared to sham treatment (p=0.0303, FIG. 9D). The increased expression of CD59 and vitronectin can provide additional protection to corneal cells from complement-induced cell death.

In summary, the presently disclosed results showed that CSSC secretome induces corneal wound healing by dampening inflammation, reducing fibrotic ECM deposition, enhancing sensory neuron regeneration, and preventing cell death by proteins involved in cell-cell adhesion, wound healing, neuroprotection, and complement inhibition (FIG. 10).

### Discussion

In the present example, a mouse corneal wound model was used to exhibit the therapeutic effect of CSSC secretome on promoting scarless wound healing and increasing sensory nerve regeneration. Further, the present example shows that inhibition of complement systems plays a role in scarless wound healing. Proteomic data indicated some important proteins in the CSSC secretome involved in the process, such as CD59, C1QBP, vitronectin, SERPING1 in promoting corneal wound healing; TIMP1, TIMP2, SEMA5A in promoting neuronal regeneration; NEO1 and APP in neuron projection development and axon guidance.

The CSSC used herein expressed the stem cell markers CD90, CD73, CD105, OCT4, and ABCG2 indicating their stemness. Previously, it was reported the potential of CSSC to promote corneal wound healing in a variety of corneal scarring models (Du et al., Stem Cells 27, 1635-1642 (2009); Basu et al., Sci Transl Med 6, 266ra172 (2014); Weng et al., Eye Vis (Lond) 7, 52 (2020); Ghoubay et al., Stem Cells Transl Med 9, 917-935 (2020); Vereb et al., Sci Rep 6, 26227 (2016); Shojaati et al., Stem Cells Transl Med 8, 11921201 (2019)). Further, reports have explored the role of stem cell-derived paracrine factors and extracellular vesicles (EVs) for corneal wound healing by upregulation of Akt signaling pathway (Leszczynska et al., Sci Rep 8, 15173 (2018)) or by delivery of miRNAs (Shojaati et al., Stem Cells Transl Med 8, 11921201 (2019)). There have been sparse studies reporting stem cell secretome which spans both EVs and soluble proteins present in cells' secretion cargo.

The present example indicates that secretome alone can recapitulate the wound healing effect of CSSC and it can be a better approach for corneal wound healing due to the non-involvement of the cellular part. Also, CSSC secretome contains multiple proteins involved in corneal wound healing and cell adhesion, such as CD59, C1QBP, vitronectin, and SERPING1. During corneal wounds, corneal epithelial cells disassemble their hemidesmosomes and migrate as a sheet to cover the wound by activating focal contact, a type of adherent junction (Gipson et al., Acta Ophthalmol Suppl 10.1111/j.1755-3768.1992.tb02162.x, 13-17 (1992)). This junction is present only at the end of migrating cells. The upregulation of key cell adhesion proteins in CSSC secretome as compared to fibroblast secretome could be involved in increasing the adherent junctions in the migrating corneal cells and support more effective wound healing.

Inflammation is the body's active and reactive defense mechanism which stems from an efficient non-specific process of its response to a host of pathogens. This process mainly involves the infiltration of white blood cells and other immune cells including macrophages to the injured site to clear pathogens which results in symptoms such as redness, warmth, and swelling. Inflammation plays a context-dependent role; it is beneficial in vascular tissues but harmful in avascular tissues like the cornea. In response to inflammation, the cornea can develop different pathologies like superficial punctate keratitis, epithelial erosions, corneal swelling, neovascularization, etc., if left untreated (Josephson et al., J Am Optom Assoc 59, 679-685 (1988); Behrens et al., Cornca 25, 900-907 (2006); Shahsuvaryan et al., Trends Pharmacol Sci 38, 667-668 (2017)). It was previously reported that after corneal wound there is increased infiltration of CD11b+/Ly6G+ neutrophils in corneas at 24 hr which was reduced in CSSC-treated wounds (Hertsenberg et al., PLoS One 12, e0171712 (2017)). Also, CD45+/CD11b+/Ly6C+ monocytes have been reported to increase just after 6 hrs. of dulled blade wound induction in the cornca (Pal-Ghosh et al., Invest Ophthalmol Vis Sci 55, 2757-2765 (2014)). The ability of CSSC secretome to reduce the number of CD45+/CD11b+Gr+/CD11b+F4/80+ cells indicated that CSSC secretome can be an ideal candidate to reduce corneal inflammation and clear corneal opacity. One of the major complications of corneal wound healing is fibrosis which is mainly mediated by transforming growth factor (TGF-B), which is involved in myofibroblast conversion and fibrotic ECM formation (Ljubimov et al., Prog Retin Eye Res 49, 17-45 (2015); Terai et al., Invest Ophthalmol Vis Sci 52, 8208-8215 (2011)). Although there are a few anti-TGF-β drugs approved for human use like ROCK inhibitors (Chen et al., Invest Ophthalmol Vis Sci 50,3662-3670 (2009)), Rosiglitazone (Karamichos et al., Exp Eye Res 124, 31-36 (2014)), and Trichostatin A (Sharma et al., Invest Ophthalmol Vis Sci 50, 2695-2701 (2009)), a biologic treatment which can become part of routine clinical practice to prevent fibrotic ECM and provide scarless wound healing is still far away. It was shown recently that CSSC derived secretome can reduce fibrosis markers SPARC, CTGF, and fibronectin and increase corneal wound healing in cultured human corneal fibroblasts in vitro (Kumar et al., Invest Ophthalmol Vis Sci 59, 3728-3738 (2018)). The ability of CSSC secretome to effectively reduce both inflammation and fibrosis in the current corneal wound model provides a novel treatment strategy to use CSSC secretome for effective scarless corneal wound healing.

Inflammatory cells have been reported to induce corneal opacity and the application of steroids or use of surgical techniques is often employed to reduce these cells and treat corneal opacity (Yun et al., Invest Ophthalmol Vis Sci 57, 1749-1756 (2016)). However, thesc treatments are unable to regenerate sensory neurons and complete clearance of immune cells might not be possible if there is no sensory nerve regeneration. It was shown that sensory nerve regeneration can be achieved in wounded corneas by removing the sympathetic nerve using superior cervical ganglionectomy, which can restore corneal transparency (Yun et al., Invest Ophthalmol Vis Sci 57, 1749-1756 (2016)). Notably, regeneration of sensory nerves at the last stage of corneal disease, when corneal transplantation is the only option, can rescue corncal transparency. The infiltration of immune cells in the scarred cornea can produce various pathogenic cytokines like vascular endothelial growth factor-A (VEGF-A) which could induce neovascularization and innervation of sympathetic nerve fibers in the cornea which increase corneal opacity (Yun et al., Immunity 53, 1050¬1062 c1055 (2020)). CSSC secretome effectively reduced the infiltration of inflammatory and immune cells in the wounded corneas which results in regeneration of sensory nerves, providing an effective alternative to complex surgical techniques and an easy approach to restoring corneal transparency. The detailed investigation of CSSC and fibroblast secretome disclosed herein provided novel insight into CSSC secretome-mediated neuroprotection. The plethora of proteins present in CSSC secretome providing axon guidance cues and the proteins involved in neuron projection development and differentiation might further explain the therapeutic role of CSSC secretome in corncal wound healing. Interestingly, CSSC secretome expressed 18 different axon guidance proteins whereas fibroblast secretome expressed none. These axon guidance proteins provide neuroprotection by guiding neurons to their specific targets. e.g., neogenin (NEO1) found in CSSC secretome interacts with netrin-1 and acts as an axon guidance molecule in vivo and the neurons expressing NEOI can navigate their ventral trajectory by using several attractive and repulsive cues (Wilson et al., Dev Biol 296, 485-498 (2006)). Another unique protein in CSSC secretome, amyloid precursor protein (APP) is involved in both neuron projection development and axon guidance. Although APP's role has been focused mainly on the pathogenesis of Alzheimer's disease, recent trauma research has shown the considerable neuroprotective role of APP in traumatic brain injury models (Plummer et al., Aging Dis 7, 163-179 (2016)), emphasizing it as an ideal future therapeutic candidate for neuroprotection. Collectively, CSSC secretome showed the presence of hundreds of these neuroprotective proteins which can be used as effective small molecule based therapeutic modalities individually or in combination for neuroprotection and in regenerating sensory neurons in wounded corneas to restore vision and in other optic neuropathies like glaucoma and other neurodegenerative disorders.

One of the reasons for secretome-mediated neuroprotection could be the inhibition of complements by proteins present in CSSC secretome. Complement inhibition has been shown to be neuroprotective after cerebral ischemia and reperfusion (Yang et al., J Neurochem 124, 523-535 (2013)). The complement CD59 gene encodes a ubiquitously expressed membrane-bound glycoprotein which inhibits complement system. Mechanistically, CD59, also called membrane attack complex (MAC) inhibitory protein, binds complement C8 and/or C9 during MAC formation, thus preventing cells from generating autoantibodies, which cause cell death, kill themselves, and inhibit the terminal pathway of complement cascade (Walport et al., N Engl J Med 344, 1058-1066 (2001); Bubeck et al., J Mol Biol 405, 325-330 (2011)). Reduction of CD59 after corneal wound and its increase after CSSC secretome treatment potentially reflects a novel role for CSSC secretome for protection of cell death and promotion of corneal wound healing. Vitronectin is found in serum and tissues and promotes cell adhesion and spreading, inhibits the membrane-damaging effect of the terminal cytolytic complement pathway, and binds to several serpin serine protease inhibitors. SERPING1 gene encodes a highly glycosylated plasma protein involved in the regulation of the complement cascade. SERPING1 encoded protein, C1 inhibitor, inhibits activation of C1r and C1s, which are the first complement components, and thus inhibits complements (Bos et al., J Biol Chem 278, 29463-29470 (2003)). During complement activation, C1r and C1s are associated with another complement component C1q to form the first component of the serum complement system. The C1QBP gene-encoded protein further inhibits C1 activation by binding to the globular heads of C1q molecules. Normally the cornea maintains very high levels of complement inhibitory proteins CD59, decay-accelerating factor (DAF, CD55), and membrane cofactor protein (MCP, CD46), all strongly expressed in the corneal epithelium and also to somewhat extent in corneal stroma (Bora et al., Invest Ophthalmol Vis Sci 34, 3579-3584 (1993)). Loss of these proteins after corneal wound might lead to activation of complements and increased cell death. CD59 is a cell surface protein and is attached to the cell membrane by a glycosylphosphatidylinositol (GPI) tail. CD59 is the only membrane-bound inhibitor of the terminal pathway of complement cascade and prevents self-destruction of cells. Recently, complement modulation has been shown to reverse the pathology of retinal pigment epithelial cells in age-related macular degeneration (Cerniauskas et al., Stem Cells Transl Med 9, 1585-1603 (2020)). These results suggest that complement inhibition could be equally beneficial in promoting wound healing and is one of the important mechanisms by which CSSC secretome induces corneal wound healing and neuroprotection. It was recently reported the role of a different stem cell type, trabecular meshwork stem cell secretome (TMSC) in the protection of retinal ganglion cells in ocular hypertension glaucoma mouse models (Kumar et al., Biorxiv 10.1101/2021.06.18.449038 (2021)). Since CSSC secretome also harbors these neuroprotective proteins, it could be equally effective for the treatment of other optic neuropathies like glaucoma.

In this example, the therapeutic role of corneal stromal stem cell (CSSC) secretome for scarless corneal wound healing and corneal sensory nerve rescuing is reported. The present example uncovered that CSSC secretome dampens inflammation, reduces fibrosis, induces sensory nerve regeneration, and rescues corneal cells by inhibiting the complement system in the wounded mouse corneas. This example also provides pre-clinical evidence for the use of CSSC secretome as a biologic treatment for corneal scarring to prevent corneal blindness. Here, it is delineated a plethora of proteins in the CSSC secretome, which individually or in combination have the potential as future therapies for scarless corneal wound healing.

Although the presently disclosed subject matter and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein can be utilized according to the presently disclosed subject matter. Accordingly, the appended claims are intended to include within their scope such processes, machines, manufacture, compositions of matter, means, methods, or steps.

Patents, patent applications, publications, product descriptions and protocols are cited throughout this application the disclosures of which are incorporated herein by reference in their entireties for all purposes.

What is claimed is:

1. A pharmaceutical composition comprising at least one complement inhibitory protein.

2. The pharmaceutical composition of claim 1, wherein the complement inhibitory protein comprises a CD59 polypeptide or a functional fragment thereof.

3. The pharmaceutical composition of claim 1, wherein the complement inhibitory protein comprises a SERPING1 polypeptide or a functional fragment thereof.

4. The pharmaceutical composition of claim 1, wherein the complement inhibitory protein comprises a vitronectin (VTN) polypeptide or a functional fragment thereof.

5. The pharmaceutical composition of claim 1, wherein the complement inhibitory protein comprises a C1QBP polypeptide or a functional fragment thereof.

6. The pharmaceutical composition of claim 2, wherein the CD59 polypeptide comprises an amino acid sequence at least 80% identical to the amino acid sequence set forth in SEQ ID NO: 1 or SEQ ID NO: 2.

7. The pharmaceutical composition of claim 3, wherein the SERPING1 polypeptide comprises an amino acid sequence at least 80% identical to the amino acid sequence set forth in SEQ ID NO: 3 or SEQ ID NO: 4.

8. The pharmaceutical composition of claim 4, wherein the VTN polypeptide comprises an amino acid sequence at least 80% identical to the amino acid sequence set forth in SEQ ID NO: 5, SEQ ID NO: 6, SEQ ID NO: 7, or SEQ ID NO: 8.

9. The pharmaceutical composition of claim 5, wherein the C1QBP polypeptide comprises an amino acid sequence at least 80% identical to the amino acid sequence set forth in SEQ ID NO: 9 or SEQ ID NO: 10.

10. The pharmaceutical composition of claim 1, further comprising at least one polypeptide selected from the group consisting of TIMP1, TIMP2, SEMA5A, FKBP4, VCL, LGALS1, CTSZ, PQBP1, TGFB1, LAMB1, PRPF19, LEPRE1, LRP1, VASP, YWHAH, SERPINF1, JAG1, GPC1, YWHAE, IQGAP1, ARHGDIA, SOD1, THY1, APP, DBN1, MAP1B, AXL, TWF2, COL3A1, ALCAM, YWHAG, DPYSL2, CALR, CSF1, FLRT2, CAPRIN1, NEO1, EPB41L3, DPYSL3, APOD, HMGB1, VAPA, CRKL, FN1, PDLIM7, SPTBN1, MAP4, ENAH, EZR, PTPRK, FLNA, HSP90AB1, SPTAN1, FAM129B, CTTN, ACTR2, EXT1, POSTN, XRCC5, SPOCK1, ITGB1, TRIOBP, LAMB2, RHOA, PRMT1, STMN1, SERPINE2, EIF4G1, DBNL, PTK7, WDR1, CFL1, MANF, DAG1, VIM, B2M, LDLR, VEGFA, YAP1, VEGFC, PDLIM5, CRABP2, and SPON2.

11. The pharmaceutical composition of claim 10, wherein the at least one polypeptide comprises a TIMP1 polypeptide or a functional fragment thereof.

12. The pharmaceutical composition of claim 11, wherein the TIMP1 polypeptide comprises an amino acid sequence at least 80% identical to the amino acid sequence set forth in SEQ ID NO: 11.

13. The pharmaceutical composition of claim 10, wherein the at least one polypeptide comprises a TIMP2 polypeptide or a functional fragment thereof.

14. The pharmaceutical composition of claim 13, wherein the TIMP2 polypeptide comprises an amino acid sequence at least 80% identical to the amino acid sequence set forth in SEQ ID NO: 12.

15. The pharmaceutical composition of claim 10, wherein the at least one polypeptide comprises a SEMA5A polypeptide or a functional fragment thereof.

16. The pharmaceutical composition of claim 15, wherein the SEMA5A polypeptide comprises an amino acid sequence at least 80% identical to the amino acid sequence set forth in SEQ ID NO: 13.

17. The pharmaceutical composition of claim 1, further comprising at least one polypeptide selected from the group consisting of NEO1, APP, FKBP4, VCL, LGALS1, CTSZ, PQBP1, TGFB1, LAMB1, PRPF19, LEPRE1, LRP1, VASP, YWHAH, SERPINF1, JAG1, TIMP2, GPC1, YWHAE, IQGAP1, ARHGDIA, SOD1, THY1, APP, DBN1, MAP1B, AXL, TWF2, COL3A1, ALCAM, YWHAG, DPYSL2, CALR, CSF1, FLRT2, CAPRIN1, EPB41L3, DPYSL3, APOD, HMGB1, VAPA, CRKL, FN1, PDLIM7, SPTBN1, MAP4, ENAH, EZR, PTPRK, FLNA, HSP90AB1, SPTAN1, FAM129B, CTTN, ACTR2, EXT1, POSTN, SEMASA, XRCC5, SPOCK1, ITGB1, TRIOBP, LAMB2, RHOA, PRMT1, STMN1, SERPINE2, EIF4G1, DBNL, PTK7, WDR1, CFL1, MANF, DAG1, VIM, B2M, LDLR, VEGFA, YAP1, VEGFC, PDLIM5, CRABP2, and SPON2.

18. The pharmaceutical composition of claim 1, further comprising at least one polypeptide selected from the group consisting of DCN, APOH, TIMP1, PLAT, TGFB1, C1QBP, COLIA1, LOX, SPARC, PDGFRA, PDGFRB, SERPING1, ANXA5, COL1A2, AXL, COL3A1, PKM, FBLN1, PEAR1, APOD, FN1, FLNA, COL5A1, GSN, ANXA1, B4GALT1, POSTN, PRKAR1A, PDGFD, CORO1B, CD59, YWHAZ, CD44, CYR61, RHOA, CAPZB, SERPINE2, PTK7, DAG1, NACA, YAP1, ALDOA, PARK7, PRDX1, TMOD3, EEF2, YWHAZ, PFN1, FASN, EIF4H, TWF2, PLIN3, EIF2A, YWHAE, ENO1, RAN, FAM129B, PUF60, PKM, SPTAN1, AHNAK, FSCN1, BAG3, EPS15L1, S100A11, VAPA, SND1, AHSA1, CAPZB, CORO1B, VASP, KTN1, FLNB, ANXA2, RANBP1, DBNL, HNRNPK, PLEC, TNKS1BP1, RTN4, EEF1G, PKM, EEF1D, CAST, TAGLN2, YWHAB, CAPG, RPL29, LDHA, HSP90AB1, CALD1, CAST, SERBP1, PRDX6, CALD1, MAPRE1, NUDC, IQGAP1, CRKL, TWF1, PDLIM5, PAICS, EIF4G1, IDH1, HSPA8, PDLIM1, PCBP1, SPTBN1, RPL14, CTTN, VASN, RDX, DBN1, and DSG.

19. A pharmaceutical composition comprising a secretome harvested from a human corneal stromal stem cell (CSSC).

20. A method of preventing, reducing the risk of, and/or treating corneal scarring in a subject in need thereof, comprising administering an effective amount of a pharmaceutical composition of claim 1.
